(19) United States
(12) Patent Application Publication
(10) Pub. No.: US 2009/0247618 A1
(43) Pub. Date: Oct. 1, 2009

(54) PROCESS FOR PREPARATION OF BENZO-FUSED HETEROARYL DERIVATIVES

(76) Inventors: Scott A. Ballentine, Spring House, PA (US); Laura Reany, Spring House, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/055,924

(22) Filed: Mar. 26, 2008

(51) Int. Cl. A61K 31/357; C07D 319/16; A61P 25/08

(52) U.S. Cl. 514/452; 549/362; 549/366

(57) ABSTRACT

The present invention is directed to processes for the preparation of benzo-fused heteroaryl derivatives, useful for the treatment of epilepsy and related disorders. The present invention is further directed to processes for the preparation of intermediates in the synthesis of the benzo-fused heteroaryl derivatives.

### FIELD OF THE INVENTION

The present invention is directed to processes for the preparation of benzo-fused heteroaryl derivatives, useful for the treatment of epilepsy and related disorders. The present invention is further directed to processes for the preparation of intermediates in the synthesis of the benzo-fused heteroaryl derivatives.

### BACKGROUND OF THE INVENTION

Epilepsy describes a condition in which a person has recurrent seizures due to a chronic, underlying process. Epilepsy refers to a clinical phenomenon rather than a single disease entity, since there are many forms and causes of epilepsy. Using a definition of epilepsy as two or more unprovoked seizures, the incidence of epilepsy is estimated at approximately 0.3 to 0.5 percent in different populations throughout the world, with the prevalence of epilepsy estimated at 5 to 10 people per 1000.

An essential step in the evaluation and management of a patient with a seizure is to determine the type of seizure that has occurred. The main characteristic that distinguishes the different categories of seizures is whether the seizure activity is partial (synonymous with focal) or generalized.

Partial seizures are those in which the seizure activity is restricted to discrete areas of the cerebral cortex. If consciousness is fully preserved during the seizure, the clinical manifestations are considered relatively simple and the seizure is termed a simple-partial seizure. If consciousness is impaired, the seizure is termed a complex-partial seizure. An important additional subgroup comprises those seizures that begin as partial seizures and then spread diffusely throughout the cortex, which are known as partial seizures with secondary generalization.

Generalized seizures involve diffuse regions of the brain simultaneously in a bilaterally symmetric fashion. Absence or petit mal seizures are characterized by sudden, brief lapses of consciousness without loss of postural control. Atypical absence seizures typically include a longer duration in the lapse of consciousness, less abrupt onset and cessation, and more obvious motor signs that may include focal or lateralizing features. Generalized Tonic-clonic or grand mal seizures, the main type of generalized seizures, are characterized by abrupt onset, without warning. The initial phase of the seizure is usually tonic contraction of muscles, impaired respiration, a marked enhancement of sympathetic tone leading to increased heart rate, blood pressure, and pupillary size. After 10-20 s, the tonic phase of the seizure typically evolves into the clonic phase, produced by the superimposition of periods of muscle relaxation on the tonic muscle contraction. The periods of relaxation progressively increase until the end of the ictal phase, which usually lasts no more than 1 min. The postictal phase is characterized by unresponsiveness, muscular flaccidity, and excessive salivation that can cause stridorous breathing and partial airway obstruction. Atonic seizures are characterized by sudden loss of postural muscle tone lasting 1-2 s. Consciousness is briefly impaired, but there is usually no postictal confusion. Myoclonic seizures are characterized by a sudden and brief muscle contraction that may involve one part of the body or the entire body. (harrisonsonline.com, Mar. 29, 2001)

McComsey, D., et al. in US Patent Publication US 2006/0041008 A1, published Feb. 23, 2006 and McComsey, D., et al. in US Patent Publication US 2005/0282887 A1, published Dec. 22, 2005 disclose compounds of formula (I) and their use in the treatment of epilepsy and related disorders. McComsey, D., et al. in US Patent Publication US 2006/0041008 A1 and McComsey, D., et al. in US Patent Publication US 2005/0282887 A1 further disclose a process for the preparation of the compounds of formula (I) comprising reacting a suitable substituted amine with sulfamide.

In the process(es) as disclosed in McComsey D., et al. compounds of formula (I) wherein R¹ and R² are each hydrogen describes the use of sulfamoyl chloride (Cl—SO₂—NH₂) as a reagent, which reagent is unsuitable for large scale/commercial preparation. There remains, however, a need for a process suitable for the preparation of large scale material and/or for commercial preparation of the compounds of formula (I).

### SUMMARY OF THE INVENTION

The present invention is directed to a process for the preparation of compounds of formula (I)

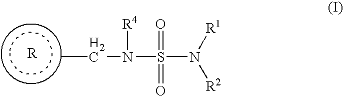

wherein

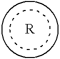

is selected from the group consisting of

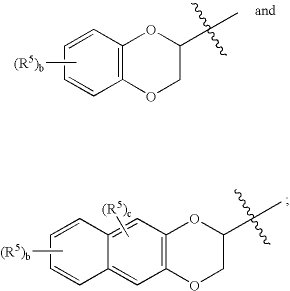

b is an integer from 0 to 4; and wherein c is an integer from 0 to 2;

each R⁵ is independently selected from the group consisting of halogen, lower alkyl and nitro;

R⁴ is selected from the group consisting of hydrogen and lower alkyl;

R¹ and R² are each independently selected from the group consisting of hydrogen and lower alkyl;

or pharmaceutically acceptable salts thereof; comprising

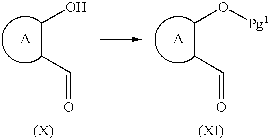

protecting a compound of formula (X) wherein

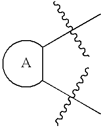

is selected from the group consisting of

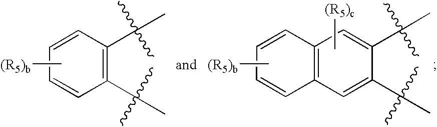

to yield the corresponding compound of formula (XI), wherein Pg¹ is an alcohol protecting group;

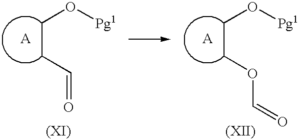

reacting the compound of formula (XI) with an oxidizing agent; in an organic solvent; to yield the corresponding compound of formula (XII);

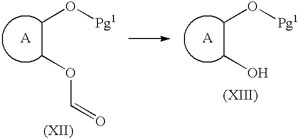

reacting the compound of formula (XII) with an organic or inorganic base; in an organic solvent, in a mixture of organic solvents or in a mixture of one or more organic solvents and water; to yield the corresponding compound of formula (XIII);

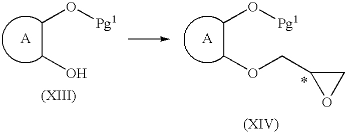

reacting the compound of formula (XIII) with a source of epoxy-methylene; in the presence of an inorganic base; at a temperature greater than about room temperature; in an organic solvent; to yield the corresponding compound of formula (XIV);

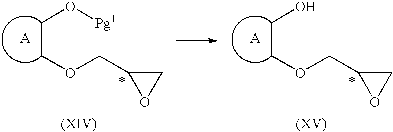

de-protecting the compound of formula (XIV); to yield the corresponding compound of formula (XV);

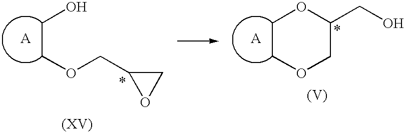

reacting the compound of formula (XV) with an organic or inorganic base; in a organic solvent; to yield the corresponding compound of formula (V);

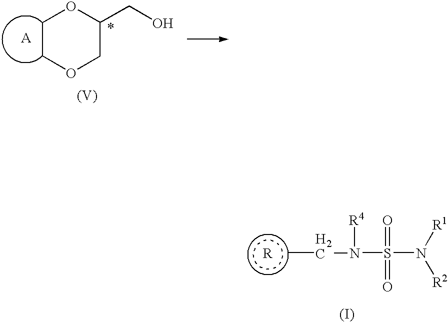

reacting the compound of formula (V); to yield the corresponding compound of formula (I).

The present invention is further directed to a process for the preparation of a compound of formula (V)

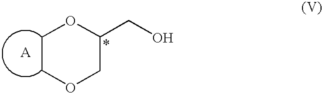

wherein

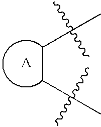

is selected from the group consisting of

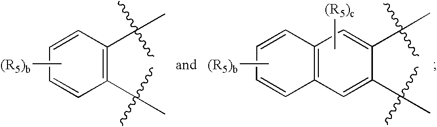

b is an integer from 0 to 4; and wherein c is an integer from 0 to 2;

each R⁵ is independently selected from the group consisting of halogen, lower alkyl and nitro;

comprising

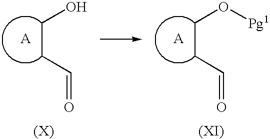

protecting a compound of formula (X); to yield the corresponding compound of formula (XI), wherein Pg¹ is an alcohol protecting group;

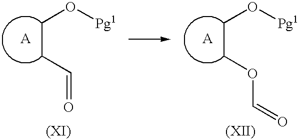

reacting the compound of formula (XI) with an oxidizing agent; in an organic solvent; to yield the corresponding compound of formula (XII);

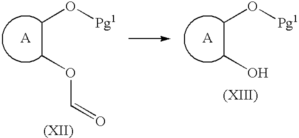

reacting the compound of formula (XII) with an organic or inorganic base; in an organic solvent, in a mixture of organic solvents or in a mixture of one or more organic solvents and water; to yield the corresponding compound of formula (XIII);

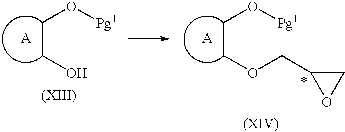

reacting the compound of formula (XIII) with a source of epoxy-methylene; in the presence of an inorganic base; at a temperature greater than about room temperature; in an organic solvent; to yield the corresponding compound of formula (XIV);

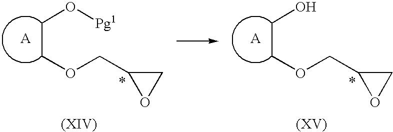

de-protecting the compound of formula (XIV); to yield the corresponding compound of formula (XV);

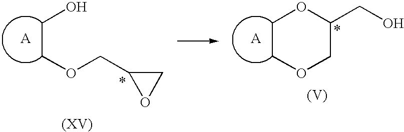

reacting the compound of formula (XV) with an organic or inorganic base; in a organic solvent; to yield the corresponding compound of formula (V).

In an embodiment, the present invention is directed to a process for the preparation of a compound of formula (I-S)

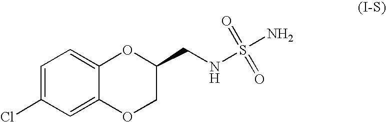

or a pharmaceutically acceptable salt thereof (wherein the compound of formula (I-S) is also known as N-[[(2S)-6-chloro-2,3-dihydro-1,4-benzodioxin-2-yl]methyl]-sulfamide); comprising

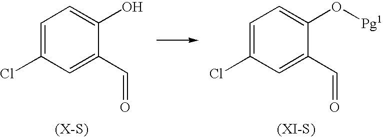

protecting a compound of formula (X-S); to yield the corresponding compound of formula (XI-S), wherein Pg¹ is an alcohol protecting group;

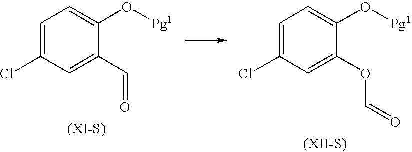

reacting the compound of formula (XI-S) with an oxidizing agent; in an organic solvent; to yield the corresponding compound of formula (XII-S);

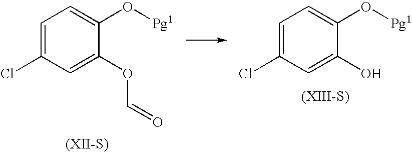

reacting the compound of formula (XII-S) with an organic or inorganic base; in an organic solvent, in a mixture of organic solvents or in a mixture of one or more organic solvents and water; to yield the corresponding compound of formula (XIII-S);

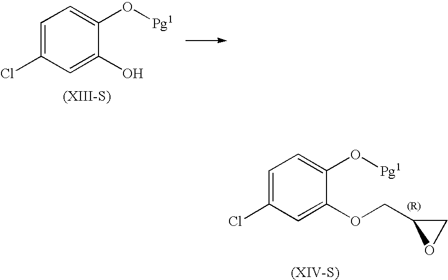

reacting the compound of formula (XIII-S) with a source of (R)-epoxy-methylene; in the presence of an inorganic base; at a temperature greater than about room temperature; in an organic solvent; to yield the corresponding compound of formula (XIV-S);

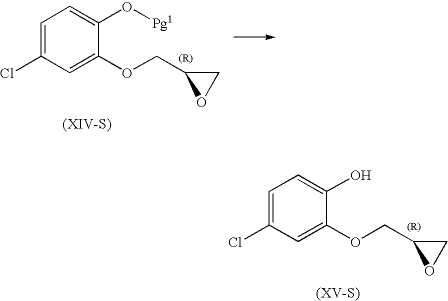

de-protecting the compound of formula (XIV-S); to yield the corresponding compound of formula (XV-S);

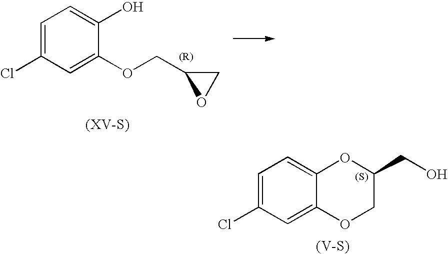

reacting the compound of formula (XV-S) with an organic or inorganic base; in a organic solvent; to yield the corresponding compound of formula (V-S);

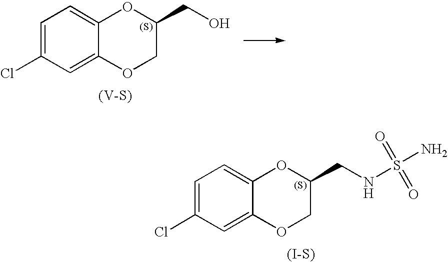

reacting the compound of formula (V-S); to yield the corresponding compound of formula (I-S).

In another embodiment, the present invention is directed to a process for the preparation of a compound of formula (V-S)

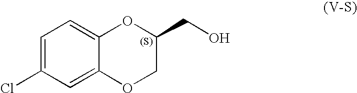

also known as (6-chloro-2,3-dihydro-benzo[1,4]dioxin-2-yl)-(S)-methanol; comprising

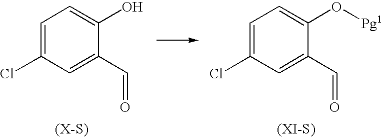

protecting a compound of formula (X-S); to yield the corresponding compound of formula (XI-S), wherein Pg¹ is an alcohol protecting group;

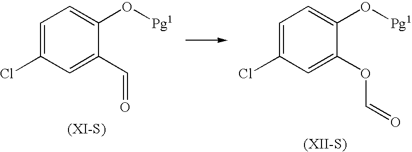

reacting the compound of formula (XI-S) with an oxidizing agent; in an organic solvent; to yield the corresponding compound of formula (XII-S);

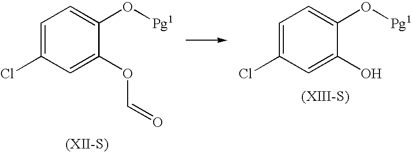

reacting the compound of formula (XII-S) with an organic or inorganic base; in an organic solvent, in a mixture of organic solvents or in a mixture of one or more organic solvents and water; to yield the corresponding compound of formula (XIII-S);

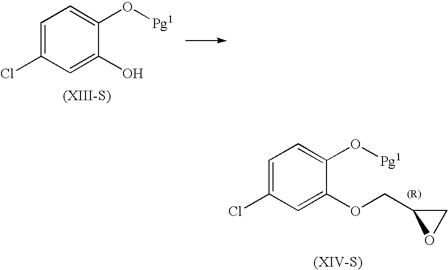

reacting the compound of formula (XIII-S) with a source of (R)-epoxy-methylene; in the presence of an inorganic base; at a temperature greater than about room temperature; in an organic solvent; to yield the corresponding compound of formula (XIV-S);

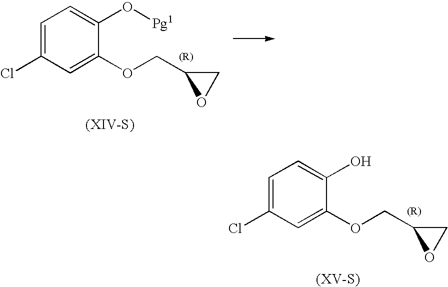

de-protecting the compound of formula (XIV-S); to yield the corresponding compound of formula (XV-S);

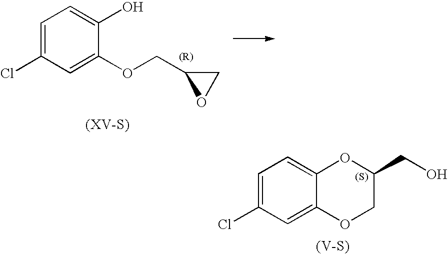

reacting the compound of formula (XV-S) with an organic or inorganic base; in a organic solvent; to yield the corresponding compound of formula (V-S).

The present invention is further directed to a process for the preparation of a compound of formula (I)

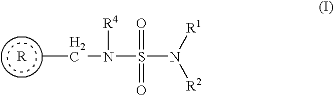

wherein

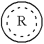

is selected from the group consisting of

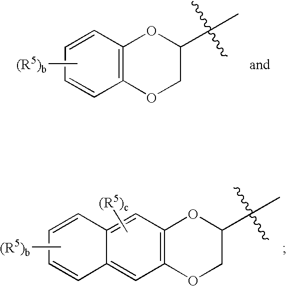

b is an integer from 0 to 4; and wherein c is an integer from 0 to 2;

each R⁵ is independently selected from the group consisting of halogen, lower alkyl and nitro;

R⁴ is selected from the group consisting of hydrogen and lower alkyl;

R¹ and R² are each independently selected from the group consisting of hydrogen and lower alkyl;

or pharmaceutically acceptable salts thereof; comprising

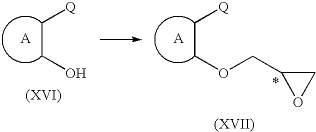

reacting a compound of formula (XVI) wherein

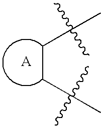

is selected from the group consisting of

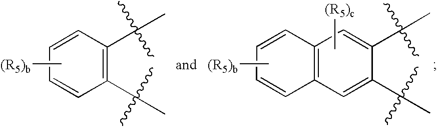

and wherein Q is selected from the group consisting of —C(O)—(C₁₋₄alkyl); with a source of epoxy-methylene; in the presence of an inorganic base; at a temperature greater than about room temperature; in an organic solvent; to yield the corresponding compound of formula (XVII);

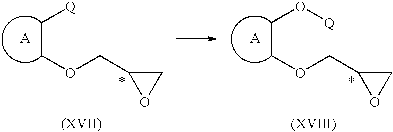

reacting the compound of formula (XVII) with an oxidizing agent; in an organic solvent; to yield the corresponding compound of formula (XVIII);

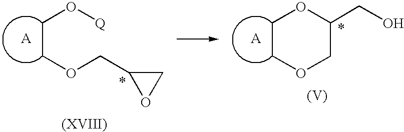

reacting the compound of formula (XVIII) with an organic or inorganic base; in a organic solvent; to yield the corresponding compound of formula (V);

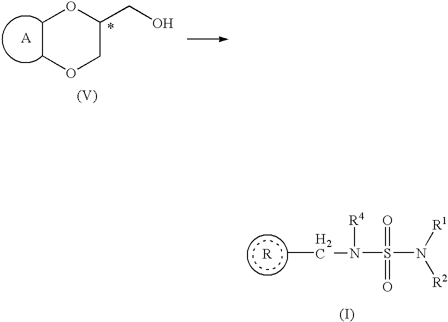

reacting the compound of formula (V); to yield the corresponding compound of formula (I).

The present invention is further directed to a process for the preparation of a compound of formula (V)

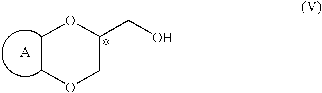

wherein

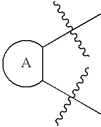

is selected from the group consisting of

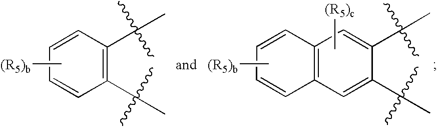

b is an integer from 0 to 4; and wherein c is an integer from 0 to 2;

each R⁵ is independently selected from the group consisting of halogen, lower alkyl and nitro;

comprising

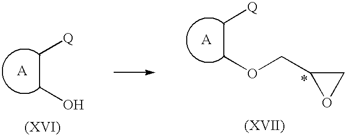

reacting a compound of formula (XVI) wherein Q is selected from the group consisting of —C(O)—(C₁₋₄alkyl); with a source of epoxy-methylene; in the presence of an inorganic base; at a temperature greater than about room temperature; in an organic solvent; to yield the corresponding compound of formula (XVII);

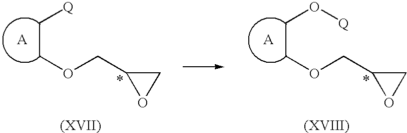

reacting the compound of formula (XVII) with an oxidizing agent; in an organic solvent; to yield the corresponding compound of formula (XVIII);

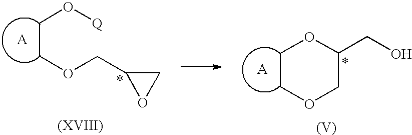

reacting the compound of formula (XVIII) with an organic or inorganic base; in a organic solvent; to yield the corresponding compound of formula (V).

In an embodiment, the present invention is directed to processes for the preparation of a compound of formula (I-S)

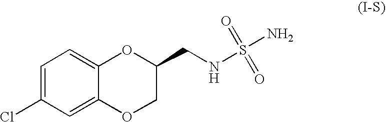

or a pharmaceutically acceptable salt thereof (wherein the compound of formula (I-S) is also known as N-[[(2S)-6-chloro-2,3-dihydro-1,4-benzodioxin-2-yl]methyl]-sulfamide); comprising

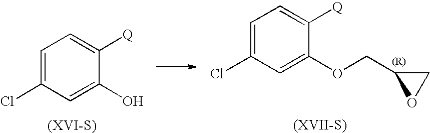

reacting a compound of formula (XVI-S) wherein Q is selected from the group consisting of —C(O)—(C₁₋₄alkyl); with a source of (R)-epoxy-methylene; in the presence of an inorganic base; at a temperature greater than about room temperature; in an organic solvent; to yield the corresponding compound of formula (XVII-S);

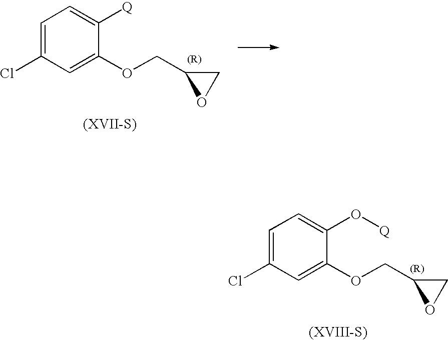

reacting the compound of formula (XVII-S) with an oxidizing agent; in an organic solvent; to yield the corresponding compound of formula (XVIII-S);

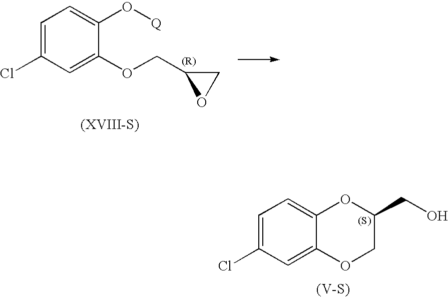

reacting the compound of formula (XVIII-S) with an organic or inorganic base; in a organic solvent; to yield the corresponding compound of formula (V-S);

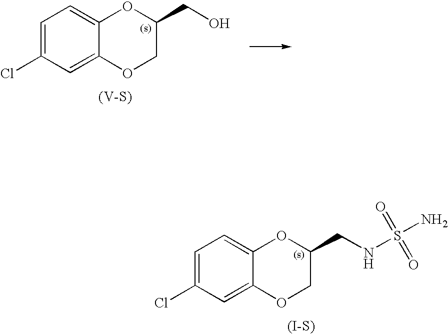

reacting the compound of formula (V-S); to yield the corresponding compound of formula (I-S).

In another embodiment, the present invention is directed to processes for the preparation of the compound of formula (V-S)

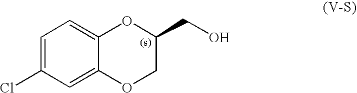

also known as (6-chloro-2,3-dihydro-benzo[1,4]dioxin-2-yl)-(S)-methanol; comprising

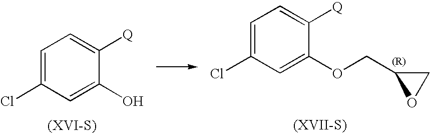

reacting a compound of formula (XVI-S) wherein Q is selected from the group consisting of —C(O)—(C₁₋₄alkyl); with a source of (R)-epoxy-methylene; in the presence of an inorganic base; at a temperature greater than about room temperature; in an organic solvent; to yield the corresponding compound of formula (XVII-S);

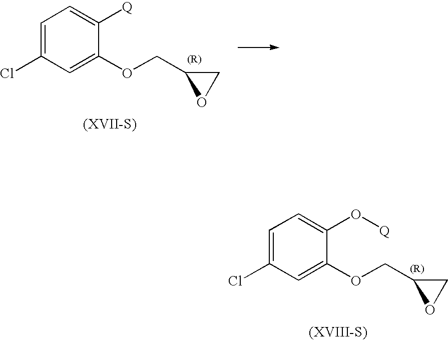

reacting the compound of formula (XVII-S) with an oxidizing agent; in an organic solvent; to yield the corresponding compound of formula (XVIII-S);

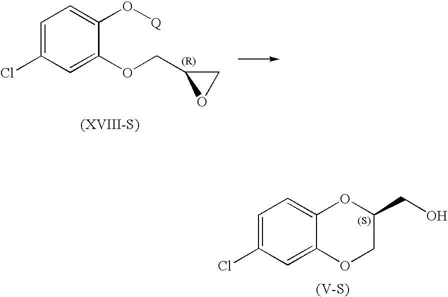

reacting the compound of formula (XVIII-S) with an organic or inorganic base; in a organic solvent; to yield the corresponding compound of formula (V-S).

The present invention is further directed to crystalline forms of the compound of formula (I-S)

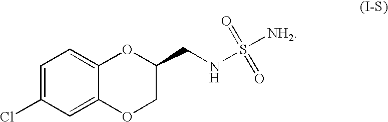

In an embodiment, the present invention is directed to crystalline form (I-SA), as hereinafter defined. In another embodiment, the present invention is directed to crystalline form (I-SB), as hereinafter defined.

The present invention is further directed to a product prepared according to any of the processes described herein.

Illustrative of the invention is a pharmaceutical composition comprising a pharmaceutically acceptable carrier and any of the crystalline forms as described herein or a product prepared according to any of the processes described herein. An illustration of the invention is a pharmaceutical composition made by mixing any of the crystalline forms as described herein or a product prepared according to any of the processes described herein and a pharmaceutically acceptable carrier. Illustrating the invention is a process for making a pharmaceutical composition comprising mixing any of the crystalline forms as described herein or a product prepared according to any of the processes described herein and a pharmaceutically acceptable carrier.

Exemplifying the invention are methods of treating epilepsy or a related disorder comprising administering to a subject in need thereof, a therapeutically effective amount of any of the compounds or pharmaceutical compositions described above.

### BRIEF DESCRIPTION OF THE FIGURES

FIG. 1 illustrates representative XRD Spectra for representative samples of Crystalline Form (I-SA) (labeled b and c) and Crystalline form (I-SB) (labeled a).

### DETAILED DESCRIPTION OF THE INVENTION

The present invention is directed to processes for the preparation of compound of formula (I)

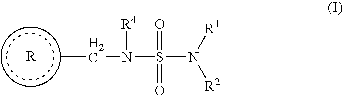

wherein R¹, R², R⁴ and

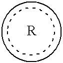

are as herein defined. The compounds of the present invention are useful in the treatment of epilepsy and related disorders.

The present invention is further directed to processes for the preparation of compounds of formula (V), which can be represented herein by either of the following formulas

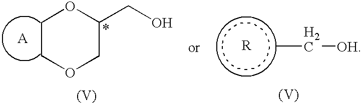

The compounds of formula (V) are useful as intermediates in the synthesis of the compounds of formula (I).

In an embodiment, the present invention is directed to processes for the preparation of the compound of formula (I-S)

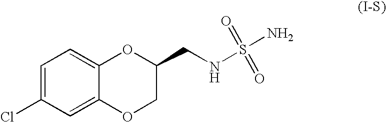

or pharmaceutically acceptable salts thereof. In another embodiment, the present invention is directed to processes for the preparation of the compound of formula (V-S)

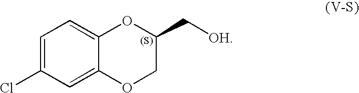

In embodiment, the present invention is directed to processes for the synthesis of compounds of formula (I-A)

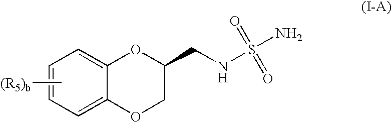

and pharmaceutically acceptable salts thereof, wherein b and R⁵ are as herein defined. Preferably, b is an integer from 0 to 2; more preferably, b is an integer from 0 to 1. Preferably R⁵ is halogen, more preferably, R⁵ is chloro.

In an embodiment of the present invention R¹ is selected from the group consisting of hydrogen and methyl. In another embodiment of the present invention R² is selected from the group consisting of hydrogen and methyl. In yet another embodiment of the present invention R¹ and R² are each hydrogen or R¹ and R² are each methyl.

In an embodiment of the present R⁴ is selected from the group consisting of hydrogen and methyl, preferably, R⁴ is hydrogen.

In an embodiment of the present invention b is an integer from 0 to 2. In another embodiment of the present invention c is an integer from 0 to 2. In another embodiment of the present invention b is an integer from 0 to 1. In another embodiment of the present invention c is an integer from 0 to 1. In yet another embodiment of the present invention the sum of b and c is an integer form 0 to 2, preferably an integer from 0 to 1. In yet another embodiment of the present invention b is an integer from 0 to 2 and c is 0.

In an embodiment of the present invention R⁵ is selected from the group consisting of halogen and lower alkyl. In another embodiment of the present invention R⁵ is selected from chloro, fluoro, bromo and methyl.

In an embodiment of the present invention,

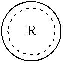

is a ring structure selected from the group consisting of

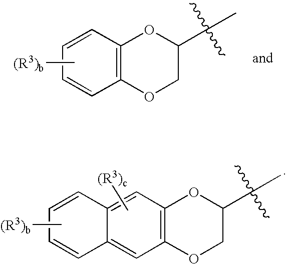

In another embodiment of the present invention,

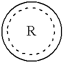

is selected from the group consisting of

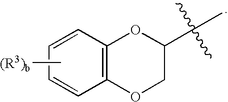

In an embodiment of the present invention,

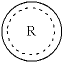

is a ring structure selected from the group consisting of 2-(6-chloro-2,3-dihydro-benzo[1,4]dioxinyl), 2-(5-chloro-2,3-dihydro-benzo[1,4]dioxinyl), 2-(7-nitro-2,3-dihydro-benzo[1,4]dioxinyl), 2-(6,7-dichloro-2,3-dihydro-benzo[1,4]dioxinyl) and 2-(2,3-dihydro-naphtho[2,3-b][1,4]dioxinyl). In another embodiment of the present invention

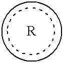

is a ring structure selected from the group consisting of 2-(5-chloro-2,3-dihydro-benzo[1,4]dioxinyl), 2-(7-nitro-2,3-dihydro-benzo[1,4]dioxinyl), 2-(6,7-dichloro-2,3-dihydro-benzo[1,4]dioxinyl) and 2-(2,3-dihydro-naphtho[2,3-b][1,4]dioxinyl).

In an embodiment of the present invention

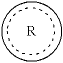

is selected from the group consisting of 2-(2,3-dihydro-benzo[1,4]dioxinyl),-2-(6-chloro-2,3-dihydro-benzo[1,4]dioxinyl), 2-(6-fluoro-2,3-dihydro-benzo[1,4]dioxinyl), 2-(5-fluoro-2,3-dihydro-benzo[1,4]dioxinyl), 2-(7-chloro-2,3-dihydro-benzo[1,4]dioxinyl), 2-(7-nitro-2,3-dihydro-benzo[1,4]dioxinyl), 2-(7-methyl-2,3-dihydro-benzo[1,4]dioxinyl), 2-(5-chloro-2,3-dihydro-benzo[1,4]dioxinyl), 2-(6-bromo-2,3-dihydro-benzo[1,4]dioxinyl), 2-(6,7-dichloro-2,3-dihydro-benzo[1,4]dioxinyl), 2-(8-chloro-2,3-dihydro-benzo[1,4]dioxinyl) and 2-(2,3-dihydro-naphtho[2,3-b][1,4]dioxinyl).

In another embodiment of the present invention,

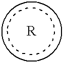

is selected from the group consisting 2-(2,3-dihydro-benzo[1,4]dioxinyl), 2-(6-chloro-2,3-dihydro-benzo[1,4]dioxinyl), 2-(7-chloro-2,3-dihydro-benzo[1,4]dioxinyl), 2-(7-methyl-2,3-dihydro-benzo[1,4]dioxinyl), 2-(6-bromo-2,3-dihydro-benzo[1,4]dioxinyl) and 2-(6,7-dichloro-2,3-dihydro-benzo[1,4]dioxinyl). In another embodiment of the present invention,

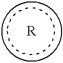

is selected from the group consisting of 2-(2,3-dihydro-benzo[1,4]dioxinyl), 2-(7-methyl-2,3-dihydro-benzo[1,4]dioxinyl) and 2-(6-bromo-2,3-dihydro-benzo[1,4]dioxinyl).

In an embodiment of the present invention, the stereo-center on the compound of formula (I) is in the S-configuration. In another embodiment of the present invention, the stereo-center on the compound of formula (I) is in the R-configuration.

In an embodiment of the present invention the compound of formula (I) is present as an enantiomerically enriched mixture, wherein the % enantiomeric enrichment (% ee) is greater than about 75%, preferably greater than about 85%, more preferably greater than about 90%, more preferably greater than about 95%, more preferably greater than about 98%, most preferably greater than about 99%.

Representative compounds of the present invention, are as listed in Tables 1 and 2, below. In Tables 1 and 2 below, the column headed “stereo” defines the stereo-configuration at the carbon atom of the heterocycle attached at the starred bond. Where no designation is listed, the compound was prepared as a mixture of stereo-configurations. Where an “R” or “S” designation is listed, the stereo-configuration was based on the enantiomerically enriched starting material.

TABLE 1
Representative Compounds of Formula (I)

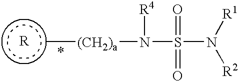

| ID No. | 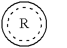 | Stereo | (CH₂)ₐ | NR⁴ | R¹ | R² |
|---|---|---|---|---|---|---|
| 1 | 2-(2,3-dihydro- | | CH₂ | NH | H | H |
| | benzo[1,4]dioxinyl) | | | | | |
| 4 | 2-(2,3-dihydro- | S | CH₂ | NH | H | H |
| | benzo[1,4]dioxinyl) | | | | | |
| 5 | 2-(2,3-dihydro- | R | CH₂ | NH | H | H |
| | benzo[1,4]dioxinyl) | | | | | |
| 6 | 2-(2,3-dihydro- | | CH₂ | NH | methyl | methyl |
| | benzo[1,4]dioxinyl) | | | | | |
| 7 | 2-(2,3-dihydro- | | CH₂ | N(CH₃) | H | H |
| | benzo[1,4]dioxinyl) | | | | | |
| 8 | 2-(6-chloro-2,3-dihydro- | S | CH₂ | NH | H | H |
| | benzo[1,4]dioxinyl) | | | | | |
| 9 | 2-(6-fluoro-2,3-dihydro- | S | CH₂ | NH | H | H |
| | benzo[1,4]dioxinyl) | | | | | |
| 13 | 2-(5-fluoro-2,3-dihydro- | S | CH₂ | NH | H | H |
| | benzo[1,4]dioxinyl) | | | | | |
| 14 | 2-(7-chloro-2,3-dihydro- | S | CH₂ | NH | H | H |
| | benzo[1,4]dioxinyl) | | | | | |
| 18 | 2-(7-nitro-2,3-dihydro- | S | CH₂ | NH | H | H |
| | benzo[1,4]dioxinyl) | | | | | |
| 19 | 2-(7-methyl-2,3-dihydro- | S | CH₂ | NH | H | H |
| | benzo[1,4]dioxinyl) | | | | | |
| 20 | 2-(5-chloro-2,3-dihydro- | S | CH₂ | NH | H | H |
| | benzo[1,4]dioxinyl) | | | | | |
| 22 | 2-(8-methoxy-2,3-dihydro- | S | CH₂ | NH | H | H |
| | benzo[1,4]dioxinyl) | | | | | |
| 24 | 2-(6-bromo-2,3-dihydro- | S | CH₂ | NH | H | H |
| | benzo[1,4]dioxinyl) | | | | | |
| 29 | 2-(6,7-dichloro-2,3-dihydro- | S | CH₂ | NH | H | H |
| | benzo[1,4]dioxinyl) | | | | | |
| 30 | 2-(8-chloro-2,3-dihydro- | S | CH₂ | NH | H | H |
| | benzo[1,4]dioxinyl) | | | | | |
| 33 | 2-(2,3-dihydro- | S | CH₂ | NH | H | H |
| | naphtho[2,3-b][1,4]dioxinyl) | | | | | |

Additional embodiments of the present invention, include those wherein the substituents selected for one or more of the variables defined herein (e.g. R¹, R², R⁴, b and R⁵, etc.) are independently selected to be any individual substituent or any subset of substituents selected from the complete list as defined herein.

As used herein, unless otherwise noted, “halogen” shall mean chlorine, bromine, fluorine and iodine.

As used herein, unless otherwise noted, the term “alkyl” whether used alone or as part of a substituent group, includes straight and branched chains. For example, alkyl radicals include methyl, ethyl, propyl, isopropyl, butyl, isobutyl, sec-butyl, t-butyl, pentyl and the like. Unless otherwise noted, “lower” when used with alkyl means a carbon chain composition of 1-4 carbon atoms.

As used herein, unless otherwise noted, “alkoxy” shall denote an oxygen ether radical of the above described straight or branched chain alkyl groups. For example, methoxy, ethoxy, n-propoxy, sec-butoxy, t-butoxy, n-hexyloxy and the like.

As used herein, the notation “*” shall denote the presence of a stereogenic center.

As used herein, unless otherwise noted, the term “enantiomerically enriched” when used to describe a compound with one stereogenic center, shall mean that one stereo-configuration of the compound is present in a greater amount than the opposite stereo-configuration of said compound. Preferably, when the compound is said to be enantiomerically enriched, the desired enantiomer of said compound is present in an enantiomeric excess of at least about 75 percent ee, more preferably at least 85 percent ee, more preferably at least 90 percent ee, more preferably at least 95 percent ee, more preferably at least 98 percent ee, most preferably at least 99 percent ee.

Abbreviations used in the specification, particularly the Schemes and Examples, are as follows:

| | |
|---|---|
| DCE = | Dichloroethane |
| DCM = | Dichloromethane |
| DIPEA or DIEA = | Diisopropylethylamine |
| DMF = | N,N-Dimethylformamide |
| DMSO = | Dimethylsulfoxide |
| EtOH = | Ethanol |
| m-CPBA = | 3-Chloroperoxybenzoic acid |
| MeOH = | Methanol |
| MOM = | Methoxymethyl |
| MTBE = | Methyl-t-butyl ether |
| NMP = | N-methyl-2-pyrrolidinone |
| NMR = | Nuclear Magnetic Resonance |
| Pd/C = | Palladium on Carbon Catalyst |
| SEM = | 2-(Trimethylsilyl)ethoxymethyl |
| TEA = | Triethylamine |
| THF = | Tetrahydrofuran |
| THP = | Tetrahydropyranyl |
| TMS = | Trimethylsilyl |
| XRD = | X-ray Diffraction |

As used herein, unless otherwise noted, the terms “epilepsy and related disorders” or “epilepsy or related disorder” shall mean any disorder in which a subject (preferably a human adult, child or infant) experiences one or more seizures and/or tremors. Suitable examples include, but are not limited to, epilepsy (including, but not limited to, localization-related epilepsies, generalized epilepsies, epilepsies with both generalized and local seizures, and the like), seizures as a complication of a disease or condition (such as seizures associated with encephalopathy, phenylketonuria, juvenile Gaucher's disease, Lundborg's progressive myoclonic epilepsy, stroke, head trauma, stress, hormonal changes, drug use or withdrawal, alcohol use or withdrawal, sleep deprivation, and the like), essential tremor, restless limb syndrome, and the like. Preferably, the disorder is selected from epilepsy (regardless of type, underlying cause or origin), essential tremor or restless limb syndrome, more preferably, the disorder is epilepsy (regardless of type, underlying cause or origin) or essential tremor.

The term “subject” as used herein, refers to an animal, preferably a mammal, most preferably a human, who is or has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

One skilled in the art will recognize that, where not otherwise specified, the reaction step(s) in the specification and claims are performed under suitable conditions (e.g. temperature, pressure, with appropriate solvents and/or reactants), according to known methods, to provide the desired product. The term “suitable conditions” shall mean a reaction step is performed under appropriate conditions (e.g. temperature, pressure, with appropriate solvents and/or reactants) according to known methods to provide the desired product.

One skilled in the art will also recognize that, in the specification and claims as presented herein, wherein a reagent or reagent class/type/ (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the term “aprotic solvent” shall mean any solvent that does not yield a proton. Suitable examples include, but are not limited to DMF, dioxane, THF, acetonitrile, pyridine, dichloroethane, dichloromethane, MTBE, toluene, and the like.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein, unless otherwise noted, the term “alcohol protecting group” shall mean a group which may be attached to the oxygen of a hydroxy group (OH)) to protect said hydroxy group from participating in a reaction, and which may be readily removed following the reaction. Suitable alcohol protecting groups include, but are not limited to, t-butyl-dimethylsilyl, trimethylsilyl (TMS), MOM, ethoxyethyl, THP, SEM, benzyl, 4-nitrobenzyl, 4-methoxybenzyl, allyl, and the like. Other suitable alcohol protecting groups may be found in texts such as T. W. Greene & P. G. M. Wuts, Protective Groups in Organic Synthesis, John Wiley & Sons, 1991, which is herein incorporated by reference in its entirety.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 75%, more preferably, the enantiomer is present at an enantiomeric excess of greater than or equal to about 85%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 75%, more preferably, the diastereomer is present at an diastereomeric excess of greater than or equal to about 85%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable base (preferably a strong base) such as NaOH, KOH, NaH, chloline hydroxide, and the like.

The present invention is directed to a process for the preparation of compounds of formula (V) as described in more detail in Scheme 1 below.

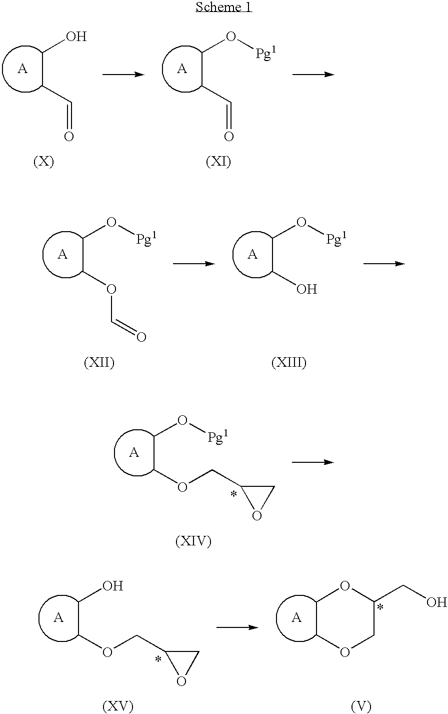

Accordingly, a suitably substituted compound of formula (X), wherein

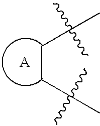

is selected from the group consisting of

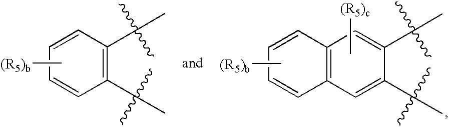

a known compound or compound prepared by known methods is protected, by reacting with a suitable protecting agent (i.e. a protecting agent stable under subsequent reaction condition, e.g. hydrolysis, oxidation) such as benzyl bromide, allyl bromide, 2-(trimethylsilyl)ethoxymethyl chloride (SEM-Cl), t-butyl-diphenylsilyl chloride, methoxy or nitro-substituted benzyl bromides (for example, 4-nitro-benzyl bromide, 4-methoxybenzyl bromide, and the like), 1-(C₁₋₄alkoxy)methyl halide or 1-(C₁₋₄alkoxy)ethyl halide, wherein the halide is Cl, Br or I (for example, MOM-Cl, ethoxyethylchloride, and the like), and the like; in the presence of an organic or inorganic base such as K₂CO₃, Na₂CO₃, Cs₂CO₃, tetramethylguanidine, TEA, and the like; preferably K₂CO₃; wherein the base is preferably present in an amount greater than or equal to about 1 molar equivalent, more preferably an excess amount; at a temperature greater than about room temperature, preferably at a temperature in the range of form about 40° C. to about 100° C., more preferably, at a temperature in the range of from about 60° C. to about 80° C., most preferably, at a temperature of about 60° C.; in an organic solvent such as DMF, THF, N-methylpyrrolidinone, and the like; to yield the corresponding compound of formula (XI), wherein Pg¹ is the corresponding alcohol protecting group. For example, wherein the protecting agent is benzyl bromide, Pg¹ is benzyl; wherein the protecting agent is allyl bromide, Pg¹ is allyl; wherein the protecting agent is MOM-Cl, Pg¹ is methoxy methyl ether (MOM). One skilled in the art will recognize that additional protecting groups and methods for incorporating said protecting groups are known in the art, as for example, described in T. W. Greene & P. G. M. Wuts, Protective Groups in Organic Synthesis, John Wiley & Sons, 1991, which is herein incorporated by reference in its entirety.

The compound of formula (XI) is reacted with a suitably selected oxidizing agent such as m-CPBA, perbenzoic acid, peracetic acid, monomagnesium peroxyphthalate and the like, preferably m-CPBA; wherein the oxidizing agent is present in an amount of at least about 1 molar equivalent, preferably about 1 to 2 molar equivalents; in an organic solvent such as DCE, DCM, chloroform, acetonitrile, NMP, and the like, preferably DCM; preferably, at about room temperature; to yield the corresponding compound of formula (XII).

The compound of formula (XII) is reacted with an organic or inorganic base such as NaOCH₃, K-t-butoxide, sodium carbonate, potassium bicarbonate, and the like, preferably NaOCH₃; wherein the base is preferably present in an amount in the range of from about 1 to about 5 molar equivalents, more preferably in an amount in the range of from about 2 to about 3 molar equivalents; in an organic solvent or mixture thereof such as methanol, ethanol, propanol, a mixture of THF and an alcohol, and the like, or in a mixture of one or more organic solvents and water; preferably in an alcohol, more preferably in methanol; preferably at a temperature in the range of from about room temperature to about reflux temperature, more preferably at about room temperature; to yield the corresponding compound of formula (XIII).

The compound of formula (XIII) is reacted with is reacted with a source of epoxy-methylene (i.e.

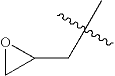

) such as glycidyl-m-nosylate, glycidyl-tosylate, epichlorohydrin, epibromohydrin, and the like, preferably glycidyl-m-nosylate or glycidyl-tosylate, preferably enantiomerically enriched source of epoxy-methylene, more preferably (R)-glycidyl-m-nosylate or (R)-glycidyl-tosylate; wherein the source of epoxy-methylene is preferably present in an amount in an amount in the range of from about 1 to about 5 molar equivalents, more preferably present in an amount in an amount in the range of from about 1 to about 2 molar equivalents, more preferably in an amount in the range of from about 1.1 to about 1.5 molar equivalent; in the presence of an inorganic base such as K₂CO₃, Na₂CO₃, Cs₂CO₃, NaH, KH, and the like, preferably K₂CO₃; wherein the inorganic base is preferably present in an amount greater than or equal to about 1 molar equivalent, more preferably, an excess amount; at a temperature greater than about room temperature, preferably at a temperature in the range of form about room temperature to about 100° C., more preferably, at a temperature in the range of from about 40° C. to about 60° C., most preferably, at a temperature of about 40° C.; in an organic solvent such as DMF, THF, N-methylpyrrolidinone, and the like, preferably DMF; to yield the corresponding compound of formula (XIV).

The compound of formula (XIV) is de-protected according to known methods, to yield the corresponding compound of formula (XV). For example, wherein the compound of formula (XIV) Pg¹ is benzyl, allyl, and the like, the compound of formula (XIV) may be de-protected by reacting with hydrogen or a source of hydrogen, preferably with hydrogen gas in the presence of a catalyst such as Pd/C, Pt, Pd(sulfide)/C, and the like; wherein the hydrogen gas is introduced at a pressure in the range of from about 10 psi to about 15 psi; in an organic solvent such as ethyl acetate, THF, isopropyl acetate, 2-methyl-THF, methyl-t-butyl ether, ethanol, and the like. Wherein Pg¹ is SEM or a silyl protecting group, the compound of formula (XIV) may be de-protected by reacting with a source of fluoride such as tetrabutylammonium fluoride, and the like, in an organic solvent such as THF, and the like. One skilled in the art will recognize that additional methods for removing protecting groups are known in the art, as for example, described in T. W. Greene & P. G. M. Wuts, Protective Groups in Organic Synthesis, John Wiley & Sons, 1991.

One skilled in the art will recognize that the reagent(s) selected for the de-protection of the compound of formula (XIV) are selected to be substantially un-reactive to the epoxy group on the compound of formula (XIV).

The compound of formula (XV) is reacted with an organic or inorganic base such as NaOCH₃, K-t-butoxide, sodium carbonate, and the like, preferably NaOCH₃; wherein the base is preferably present in an amount greater than or equal to about 1 molar equivalent, more preferably an excess amount; in a organic solvent such as methanol, ethanol, THF, acetonitrile, and the like, preferably methanol; preferably at about room temperature; to yield the corresponding compound of formula (V).

One skilled in the art will recognize that when the source of epoxy-methylene is enantiomerically enriched with one of the enantiomers, then on opening the epoxide, the compound of formula (V) is prepared as the corresponding enantiomerically enriched compound. For example, wherein enantiomerically enriched (R)-glycicyl-m-nosylate or (R)-glycidyl tosylate is reacted with the compound of formula (XIII), then the process as described in Scheme 1 above yield the corresponding compound of formula (V)

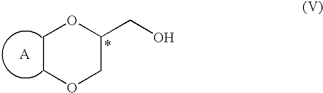

as the enantiomerically enriched (S) enantiomer at the starred (“*”) position.

Preferably, the process as described in Scheme 1 above is applied to the preparation of compounds of formula (V) wherein each R⁵ is other than nitro. One skilled in the art will recognize that wherein one or more of the R⁵ groups are nitro, the compound of formula (V) may be prepared from the corresponding compound of formula (V) wherein the substituent group at the position at which one or more nitro groups is desired, is hydrogen, by converting said hydrogen(s) to the corresponding nitro group(s) according to known methods, for example by reacting with a mixture of nitric acid and sulfuric acid, a mixture of nitric acid and acetic acid or by reacting with potassium nitrate and sulfuric acid.

The present invention is further directed to a process for the preparation of compounds of formula (V), as described in more detail in Scheme 2, below.

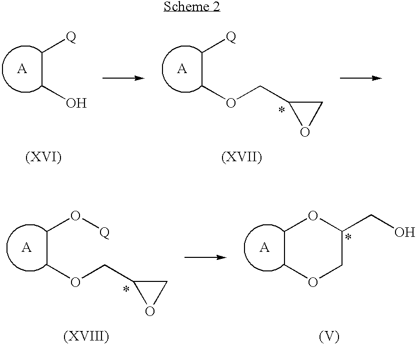

Accordingly, a suitably substituted compound of formula (XVI) wherein Q is selected from the group consisting of —C(O)—(C₁₋₄alkyl), wherein the C₁₋₄ alkyl is preferably a primary C₁₋₄alkyl, more preferably —C(O)—CH₃, and the like, a known compound or compound prepared by known methods is reacted with a source of epoxy-methylene (i.e.

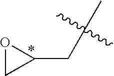

) such as glycidyl-m-nosylate, glycidyl-tosylate, epichlorohydrin, epibromohydrin, and the like, preferably glycidyl-m-nosylate or glycidyl-tosylate, preferably enantiomerically enriched source of epoxy-methylene, more preferably (R)-glycidyl-m-nosylate or (R)-glycidyl-tosylate; wherein the source of epoxy-methylene is preferably present in an amount in an amount in the range of from about 1 to about 5 molar equivalents, more preferably present in an amount in an amount in the range of from about 1 to about 2 molar equivalents, more preferably in an amount in the range of from about 1.1 to about 1.5 molar equivalent; in the presence of an inorganic base such as K₂CO₃, Na₂CO₃, Cs₂CO₃, NaH, KH, and the like, preferably K₂CO₃; wherein the inorganic base is preferably present in an amount greater than or equal to about 1 molar equivalent, more preferably, an excess amount; at a temperature greater than about room temperature, preferably at a temperature in the range of form about room temperature to about 100° C., more preferably, at a temperature in the range of from about 40° C. to about 60° C., most preferably, at a temperature of about 40° C.; in an organic solvent such as DMF, THF, N-methylpyrrolidinone, and the like, preferably DMF; to yield the corresponding compound of formula (XVII).

The compound of formula (XVII) is reacted with a suitably selected oxidizing agent such as m-CPBA, perbenzoic acid, peracetic acid, monomagnesium peroxyphthalate and the like, preferably m-CPBA; wherein the oxidizing agent is present in an amount of at least about 1 molar equivalent, preferably about 1 to 2 molar equivalents; in an organic solvent such as DCE, DCM, chloroform, acetonitrile, NMP, and the like, preferably DCM; preferably, at about room temperature; to yield the corresponding compound of formula (XVIII). Wherein Q is —C(O)—(CH₃) the compound of formula (XVII) is reacted under conditions other than with monomagnesium peroxyphthalate in DMF.

The compound of formula (XVIII) is reacted with an organic or inorganic base such as NaOCH₃, K-t-butoxide, sodium carbonate, and the like, preferably NaOCH₃; wherein the base is preferably present in an amount greater than or equal to about 1 molar equivalent, more preferably an excess amount; in a organic solvent such as methanol, ethanol, THF, acetonitrile, and the like, preferably methanol; preferably at about room temperature; to yield the corresponding compound of formula (V).

One skilled in the art will recognize that when the source of epoxy-methylene is enantiomerically enriched with one of the enantiomers, then on opening the epoxide, the compound of formula (V) is prepared as the corresponding enantiomerically enriched compound. For example, wherein enantiomerically enriched (R)-glycicyl-m-nosylate or (R)-glycidyl-tosylate is reacted with the compound of formula (XIII), then the process as described in Scheme 2 above yield the corresponding compound of formula (Va)

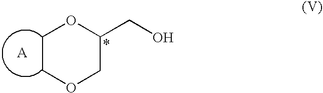

as the enantiomerically enriched (S) enantiomer at the starred (“*”) position.

The compound of formula (V) may be further reacted according to known methods (for example, as disclosed in McComsey, D., et al. in US Patent Publication US 2006/0041008 A1, published Feb. 23, 2006; McComsey, D., et al. in US Patent Publication US 2005/0282887 A1, published Dec. 22, 2005) to yield the corresponding compound of formula (I).

For example, compounds of formula (I) wherein R⁴ is hydrogen may be prepared according to the process outlined in Scheme 3.

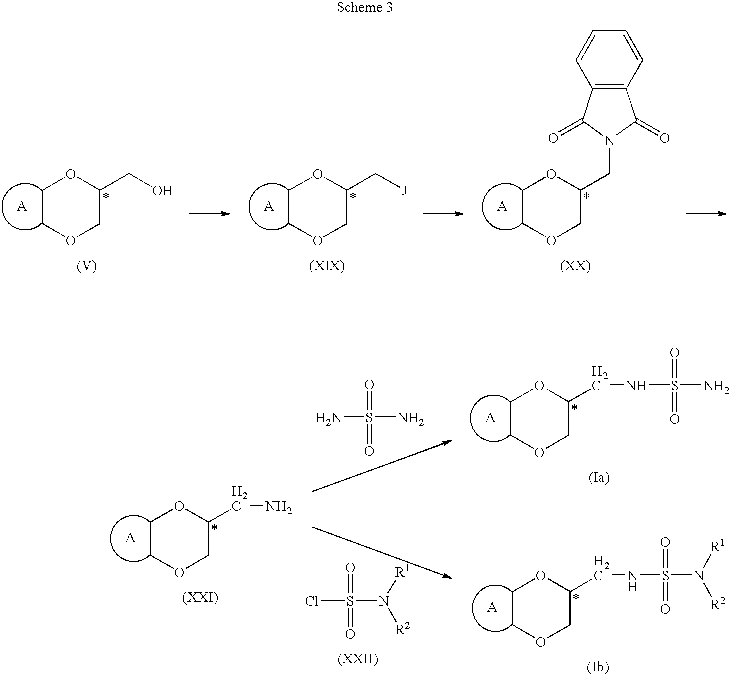

Accordingly, a suitably substituted compound of formula (V), prepared as for example outlined in Scheme 1 or 2 above, is activated, according to known method, to yield the corresponding compound of formula (XIX), wherein J is a suitable leaving group, such tosylate, Cl, Br, I, mesylate, triflate, and the like.

The compound of formula (XIX) is reacted with a phthalimide salt such as potassium phthlimide, sodium phthalimide, and the like, in an organic solvent such as DMF, DMSO, acetonitrile, and the like, preferably, at an elevated temperature in the range of from 50° C. to about 200° C., more preferably, wherein the organic solvent is DMF, DMSO and the like, at temperature in the range of from about 50° C. to about 150° C. and wherein the organic solvent is acetonitrile, and the like, at about reflux temperature, to yield the corresponding compound of formula (XX).

The compound of formula (XX) is reacted with N₂H₄, a known compound, in an organic solvent such as ethanol, methanol, and the like, preferably, at an elevated temperature in the range of from about 50° C. to about 100° C., more preferably, at about reflux temperature, and the like, to yield the corresponding compound of formula (XXI).

The compound of formula (XXI) is reacted with sulfamide (NH₂—SO₂—NH₂), a known compound, preferably wherein the sulfamide is present in an amount in the range of about 2 to about 5 equivalents, in an organic solvent such as THF, dioxane, and the like, preferably at an elevated temperature in the range of about 50° C. to about 100° C., more preferably at about reflux temperature, to yield the corresponding compound of formula (Ia), a compound of formula (I) wherein R¹ and R² are each hydrogen.

Alternatively, the compound of formula (XXI), is reacted with a suitably substituted compound of formula (XXII), a known compound or compound prepared by known methods, in the presence of a base such as TEA, DIPEA, pyridine, and the like, in an organic solvent such as DMF, DMSO, and the like, to yield the corresponding compound of formula (Ib).

In an embodiment, the present invention is directed to a process for the preparation of the compound of formula (V-S), as outlined in Scheme 4 below.

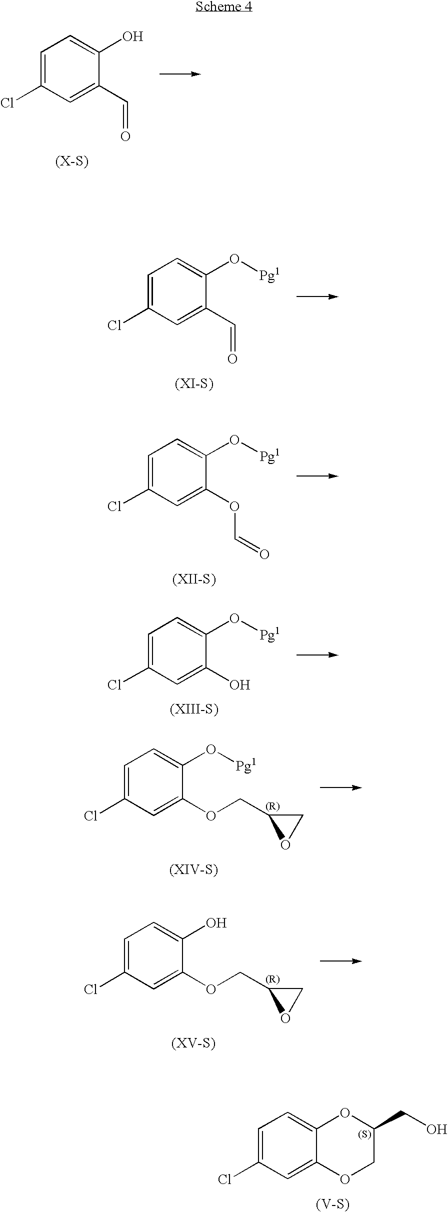

Accordingly, a suitably substituted compound of formula (X-S), a known compound or compound prepared by known methods is protected by reacting with a suitable protecting agent (i.e. a protecting agent stable under subsequent reaction condition, e.g. hydrolysis, oxidation) such as benzyl bromide, allyl bromide, 2-(trimethylsilyl)ethoxymethyl chloride (SEM-Cl), t-butyl-diphenylsilyl chloride, methoxy or nitro-substituted benzyl bromides (for example, 4-nitro-benzyl bromide, 4-methoxybenzyl bromide, and the like), 1-(C₁₋₄alkoxy)methyl halide or 1-(C₁₋₄alkoxy)ethyl halide, wherein the halide is Cl, Br or I (for example, MOM-Cl, ethoxyethylchloride, and the like), and the like; in the presence of an organic or inorganic base such as K₂CO₃, Na₂CO₃, Cs₂CO₃, tetramethylguanidine, TEA, and the like; preferably K₂CO₃; wherein the base is preferably present in an amount greater than or equal to about 1 molar equivalent, more preferably an excess amount; at a temperature greater than about room temperature, preferably at a temperature in the range of form about 40° C. to about 100° C., more preferably, at a temperature in the range of from about 60° C. to about 80° C., most preferably, at a temperature of about 60° C.; in an organic solvent such as DMF, THF, N-methylpyrrolidinone, and the like; to yield the corresponding compound of formula (XI-S), wherein Pg¹ is the corresponding alcohol protecting group. For example, wherein the protecting agent is benzyl bromide, Pg¹ is benzyl; wherein the protecting agent is allyl bromide, Pg¹ is allyl; wherein the protecting agent is MOM-Cl, Pg¹ is methoxy methyl ether. One skilled in the art will recognize that additional protecting groups and methods for incorporating said protecting groups are known in the art, as for example, described in T. W. Greene & P. G. M. Wuts, Protective Groups in Organic Synthesis, John Wiley & Sons, 1991, which is herein incorporated by reference in its entirety.

The compound of formula (XI-S) is reacted with a suitably selected oxidizing agent such as m-CPBA, perbenzoic acid, peracetic acid, monomagnesium peroxyphthalate and the like, preferably m-CPBA; wherein the oxidizing agent is present in an amount of at least about 1 molar equivalent, preferably about 1 to 2 molar equivalents; in an organic solvent such as DCE, DCM, chloroform, acetonitrile, NMP, and the like, preferably DCM; preferably, at about room temperature; to yield the corresponding compound of formula (XII-S).

The compound of formula (XII-S) is reacted with an organic or inorganic base such as NaOCH₃, K-t-butoxide, sodium carbonate, potassium bicarbonate, and the like, preferably NaOCH₃; wherein the base is preferably present in an amount in the range of from about 1 to about 5 molar equivalents, more preferably in an amount in the range of from about 2 to about 3 molar equivalents; in an organic solvent or mixture thereof such as methanol, ethanol, propanol, a mixture of THF and an alcohol, and the like, or in a mixture of one or more organic solvents and water; preferably in an alcohol, more preferably in methanol; preferably at a temperature in the range of from about room temperature to about reflux temperature, more preferably at about room temperature; to yield the corresponding compound of formula (XIII-S).

The compound of formula (XIII) is reacted with is reacted with a source of (R)-epoxy-methylene (i.e.

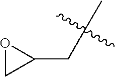

) such as (R)-glycidyl-m-nosylate, (R)-glycidyl-tosylate, (R)-epichlorohydrin, (R)-epibromohydrin, and the like, preferably (R)-glycidyl-m-nosylate or (R)-glycidyl-tosylate,; wherein the source of epoxy-methylene is preferably present in an amount in an amount in the range of from about 1 to about 5 molar equivalents, more preferably present in an amount in an amount in the range of from about 1 to about 2 molar equivalents, more preferably in an amount in the range of from about 1.1 to about 1.5 molar equivalent; in the presence of an inorganic base such as K₂CO₃, Na₂CO₃, Cs₂CO₃, NaH, KH, and the like, preferably K₂CO₃; wherein the inorganic base is preferably present in an amount greater than or equal to about 1 molar equivalent, more preferably, an excess amount; at a temperature greater than about room temperature, preferably at a temperature in the range of form about room temperature to about 100° C., more preferably, at a temperature in the range of from about 40° C. to about 60° C., most preferably, at a temperature of about 40° C.; in an organic solvent such as DMF, THF, N-methylpyrrolidinone, and the like, preferably DMF; to yield the corresponding compound of formula (XIV-S).

The compound of formula (XIV-S) is de-protected according to known methods, to yield the corresponding compound of formula (XV-S). For example, wherein the compound of formula (XIV-S) Pg¹ is benzyl, allyl, and the like, the compound of formula (XIV-S) may be de-protected by reacting with hydrogen or a source of hydrogen, preferably with hydrogen gas in the presence of a catalyst such as Pd/C, Pt, Pd(sulfide)/C, and the like; wherein the hydrogen gas is introduced at a pressure in the range of from about 10 psi to about 15 psi; in an organic solvent such as ethyl acetate, THF, isopropyl acetate, 2-methyl-THF, methyl-t-butyl ether, ethanol, and the like. Wherein Pg¹ is SEM or a silyl protecting group, the compound of formula (XIV-S) may be de-protected by reacting with a source of fluoride such as tetrabutylammonium fluoride, and the like, in an organic solvent such as THF, and the like. One skilled in the art will recognize that additional methods for removing protecting groups are known in the art, as for example, described in T. W. Greene & P. G. M. Wuts, Protective Groups in Organic Synthesis, John Wiley & Sons, 1991.

One skilled in the art will recognize that the reagent(s) selected for the de-protection of the compound of formula (XIV-S) are selected to be substantially un-reactive to the epoxy group on the compound of formula (XIV-S).

The compound of formula (XV-S) is reacted with an organic or inorganic base such as NaOCH₃, K-t-butoxide, sodium carbonate, and the like, preferably NaOCH₃; wherein the base is preferably present in an amount greater than or equal to about 1 molar equivalent, more preferably an excess amount; in a organic solvent such as methanol, ethanol, THF, acetonitrile, and the like, preferably methanol; preferably at about room temperature; to yield the corresponding compound of formula (V-S).

In another embodiment, the present invention is directed to a process for the preparation of the compound of formula (V-S), as outlined in Scheme 5 below.

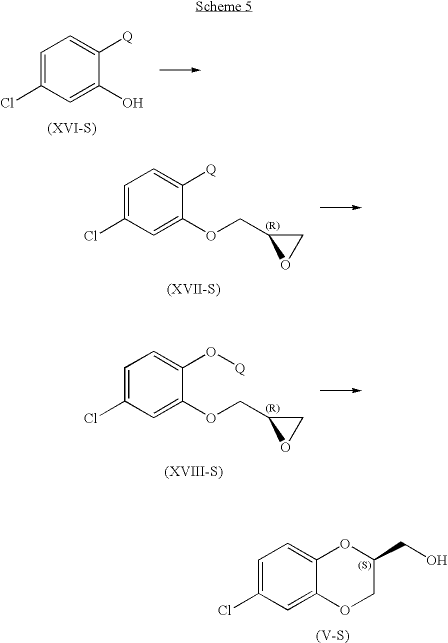

Accordingly, a suitably substituted compound of formula (XVI-S) wherein Q is selected from the group consisting of —C(O)—(C₁₋₄alkyl), wherein the C₁₋₄ alkyl is preferably a primary C₁₋₄alkyl, more preferably —C(O)—CH₃, and the like, a known compound or compound prepared by known methods is reacted with a source of (R)-epoxy-methylene (i.e.

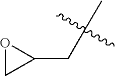

) such as (R)-glycidyl-m-nosylate, (R)-glycidyl-tosylate, (R)-epichlorohydrin, (R)-epibromohydrin, and the like, preferably (R)-glycidyl-m-nosylate or (R)-glycidyl-tosylate; wherein the source of epoxy-methylene is preferably present in an amount in an amount in the range of from about 1 to about 5 molar equivalents, more preferably present in an amount in an amount in the range of from about 1 to about 2 molar equivalents, more preferably in an amount in the range of from about 1.1 to about 1.5 molar equivalent; in the presence of an inorganic base such as K₂CO₃, Na₂CO₃, Cs₂CO₃, NaH, KH, and the like, preferably K₂CO₃; wherein the inorganic base is preferably present in an amount greater than or equal to about 1 molar equivalent, more preferably, an excess amount; at a temperature greater than about room temperature, preferably at a temperature in the range of form about room temperature to about 100° C., more preferably, at a temperature in the range of from about 40° C. to about 60° C., most preferably, at a temperature of about 40° C.; in an organic solvent such as DMF, THF, N-methylpyrrolidinone, and the like, preferably DMF; to yield the corresponding compound of formula (XVII-S).

The compound of formula (XVIII-S) is reacted with a suitably selected oxidizing agent such as m-CPBA, perbenzoic acid, peracetic acid, monomagnesium peroxyphthalate and the like, preferably m-CPBA; wherein the oxidizing agent is present in an amount of at least about 1 molar equivalent, preferably about 1 to 2 molar equivalents; in an organic solvent such as DCE, DCM, chloroform, acetonitrile, NMP, and the like, preferably DCM; preferably, at about room temperature; to yield the corresponding compound of formula (XVIII-S). Wherein Q is —C(O)—(CH₃) the compound of formula (XVII-S) is reacted under conditions other than with monomagnesium peroxyphthalate in DMF.

The compound of formula (XVIII-S) is reacted with an organic or inorganic base such as NaOCH₃, K-t-butoxide, sodium carbonate, and the like, preferably NaOCH₃; wherein the base is preferably present in an amount greater than or equal to about 1 molar equivalent, more preferably an excess amount; in a organic solvent such as methanol, ethanol, THF, acetonitrile, and the like, preferably methanol; preferably at about room temperature; to yield the corresponding compound of formula (V-S).

The compound of formula (V-S) may be further reacted to yield the corresponding compound of formula (I-S) according to known methods (for example, as disclosed in McComsey, D., et al. in US Patent Publication US 2006/0041008 A1, published Feb. 23, 2006; McComsey, D., et al. in US Patent Publication US 2005/0282887 A1, published Dec. 22, 2005).

For example, the compound of formula (I-S) may be prepared according to the process outlined in Scheme 6, below.

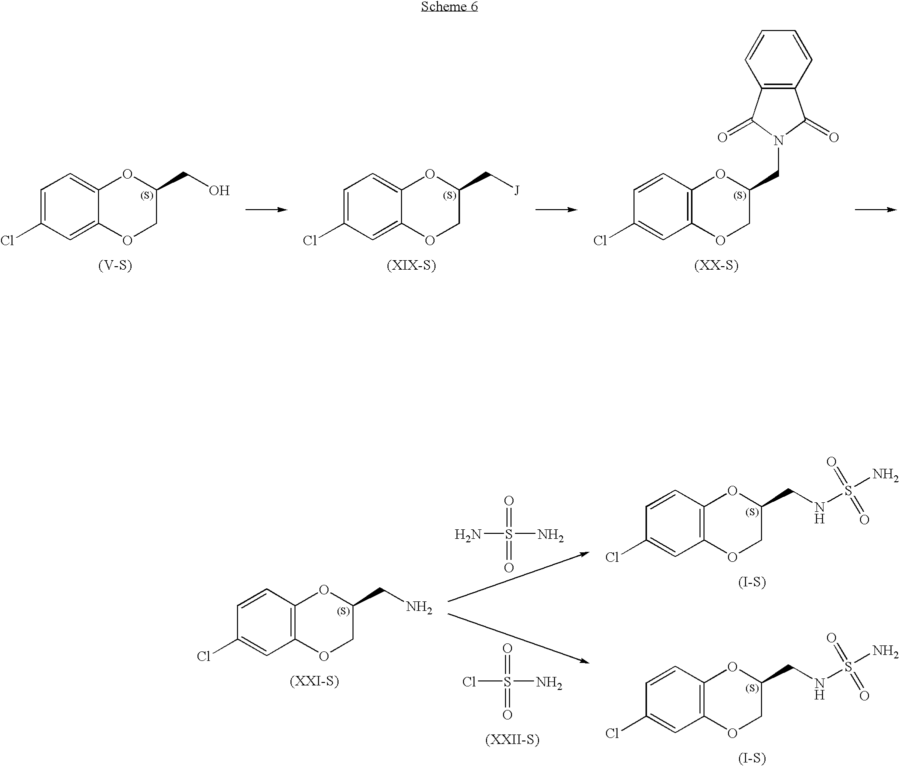

Accordingly, a suitably substituted compound of formula (V-S), prepared as for example outlined in Scheme 1, 2, 4 or 5 above, is activated, according to known method, to yield the corresponding compound of formula (XIX-S), wherein J is a suitable leaving group, such as tosylate, Cl, Br, I, mesylate, triflate, and the like.

The compound of formula (XIX-S) is reacted with a phthalimide salt such as potassium phthlimide, sodium phthalimide, and the like, in an organic solvent such as DMF, DMSO, acetonitrile, and the like, preferably, at an elevated temperature in the range of from 50° C. to about 200° C., more preferably, wherein the organic solvent is DMF, DMSO and the like, at temperature in the range of from about 50° C. to about 150° C. and wherein the organic solvent is acetonitrile, and the like, at about reflux temperature, to yield the corresponding compound of formula (XX-S).

The compound of formula (XX-S) is reacted with N₂H₄, a known compound, in an organic solvent such as ethanol, methanol, and the like, preferably, at an elevated temperature in the range of from about 50° C. to about 100° C., more preferably, at about reflux temperature, and the like, to yield the corresponding compound of formula (XXI-S).

The compound of formula (XXI-S) is reacted with sulfamide (NH₂—SO₂—NH₂), a known compound, preferably wherein the sulfamide is present in an amount in the range of about 2 to about 5 equivalents, in an organic solvent such as THF, dioxane, and the like, preferably at an elevated temperature in the range of about 50° C. to about 100° C., more preferably at about reflux temperature, to yield the corresponding compound of formula (I-S).

Alternatively, the compound of formula (XXI-S), is reacted with compound of formula (XXII-S), a known compound, also known as sulfamoyl chloride, in the presence of a base such as TEA, DIPEA, pyridine, and the like, in an organic solvent such as DMF, DMSO, and the like, to yield the corresponding compound of formula (I-S).

The present invention is further directed to crystalline forms of the compound of formula (I-S), hereinafter referred to as crystalline form (I-SA) and (I-SB). The crystalline forms of the compound of formula (I-S) may be characterized by their corresponding Powder X-ray Diffraction (PXRD) spectra.

In an embodiment, the crystalline forms of the compound of formula (I-S) may be characterized by their corresponding PXRD peaks, wherein the peaks have a relative intensity of greater than or equal to about 10% relative intensity; preferably, wherein the peaks have a relative intensity of greater than or equal to about 25% relative intensity. In an embodiment, the crystalline form of the compound of formula (I-S) may be characterized by its corresponding PXRD peaks, wherein the peaks are defined by their position (°2θ), d-spacing (Å) and relative intensity (%). In another embodiment, the crystalline form of the compound of formula (I-S) may be characterized by its corresponding PXRD peaks, wherein the peaks are defined by their position (°2θ) and d-spacing (Å).

Powder XRD spectra were measured for representative samples of the crystalline forms of the compound of formula (I-S) with peaks as listed in Tables 2 and 3 below. The PXRD spectra were measured using an X-Celerator detector, scanning form 3 to 35°2θ, at a step size of 0.0165°2θ, a time per step of 10.16 sec, an effective scan speed of 0.2067°/sec, instrument voltage of 45 kV and a current setting of 40 mA.

Crystalline form (I-SA) may be prepared for example, as described in Example 13 below. The melting point of crystalline form (I-SA) was measured for a representative sample and showed a melting point onset of 98.6° C. and a 100.8° C. Crystalline form (I-SA) may be characterized by its powder XRD peaks, as listed in Table XRD-1 below.

TABLE 2
Form (I-SA)

| Position [° 2θ] | d-spacing [Å] | Relative Intensity [%] |
|---|---|---|
| 4.50 | 19.62 | 42 |
| 15.57 | 5.69 | 13 |
| 17.38 | 5.10 | 45 |
| 18.63 | 4.76 | 100 |
| 19.97 | 4.45 | 14 |
| 20.96 | 4.24 | 22 |
| 21.62 | 4.11 | 20 |
| 22.01 | 4.04 | 11 |
| 23.97 | 3.71 | 13 |
| 25.08 | 3.55 | 21 |
| 26.91 | 3.31 | 13 |
| 28.35 | 3.15 | 12 |
| 30.76 | 2.91 | 12 |

Crystalline form (I-SB) may be prepared by re-crystallizing the compound of formula (I-S) from water, according to known methods, for example as described in Example 14 below. The melting point of crystalline form (I-SA) was measured for a representative sample and showed a melting point onset of 100.7° C. and a 102.8° C. Crystalline form (I-SB) may be characterized by its powder XRD peaks, as listed in Table 3 below.

TABLE 3
Form (I-SB)

| Position [°2θ] | d-spacing [Å] | Relative Intensity [%] |
|---|---|---|
| 4.48 | 19.74 | 100 |
| 8.91 | 9.92 | 19 |
| 13.36 | 6.62 | 7 |
| 17.84 | 4.97 | 6 |
| 18.61 | 4.77 | 4 |
| 22.33 | 3.98 | 5 |
| 26.86 | 3.32 | 28 |

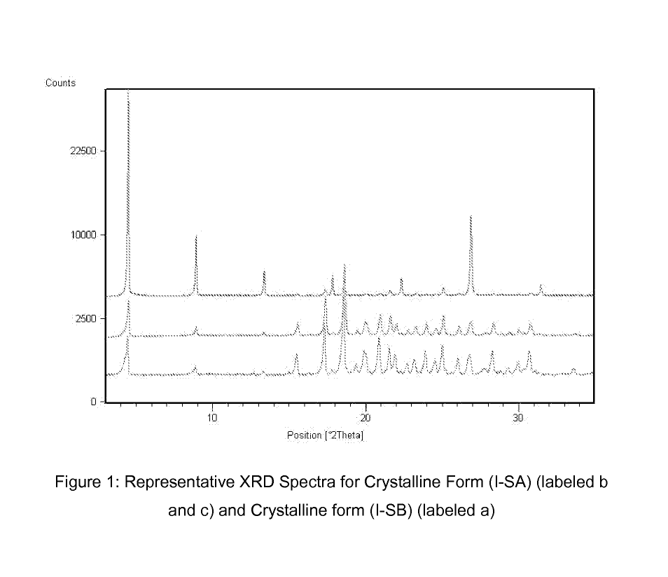

FIG. 1 illustrates representative powder X-ray diffraction patterns corresponding to the following samples: (a) the topmost scan corresponds to a representative sample of crystalline form (I-SB); (b) the middle and bottom scans correspond to two separately prepared samples of crystalline form (I-SA).

The present invention further comprises pharmaceutical compositions containing one or more of the compounds prepared according to any of the processes described herein with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 1-1000 mg and may be given at a dosage of from about 0.01-300 mg/kg/day, or any range therein, preferably from about 0.5-100 mg/kg/day, or any range therein, more preferably from about 1.0-25.0 mg/kg/day, or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating epilepsy or a related disorder described in the present invention may also be carried out using a pharmaceutical composition comprising any of the compounds as defined herein and a pharmaceutically acceptable carrier. The pharmaceutical composition may contain between about 0.1 mg and 1000 mg, preferably about 50 to 500 mg, of the compound, or any range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of epilepsy or related disorders is required.

The daily dosage of the products may be varied over a wide range from 0.01 to 10,000 mg per adult human per day, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 500.0 mg/kg of body weight per day, or any range therein. Preferably, the range is from about 0.5 to about 100.0 mg/kg of body weight per day, or any range therein, more preferably, from about 1.0 to about 50.0 mg/kg of body weight per day, or any range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### EXAMPLE 1

### 2-Benzyloxy-5-chloro-benzaldehyde

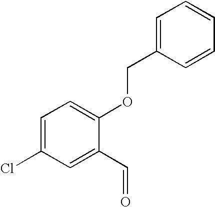

A 2 L three-necked flask (equipped with mechanical stirrer, nitrogen inlet, and a thermocouple) was charged with 5-chlorosalicylaldehyde (7, 23 g, 0.147 mol), benzyl bromide (25.1 g, 0.147 mol), potassium carbonate (20.3 g, 0.147 mol), and N,N-dimethylformamide (650 mL). The reaction mixture was heated to 60° C. for 18 h (overnight). The reaction mixture was poured into water (700 mL) and then extracted with ethyl ether (3×300 mL). The combined organics were washed with brine (2×200 mL), dried (MgSO₄), and concentrated to yield a crude product. The crude product was dissolved in 1:1 heptane-dichloromethane and loaded onto a Biotage 75S (200 g silica gel) and eluted with heptane (1 L), then 1:9 (3 L), and 3:7 (2 L) ethyl acetate-heptane to yield the title compound as a solid

¹H NMR (CDCl₃) δ ppm: 10.48 (s, 1H), 7.81 (d, J=3.1 Hz, 1H), 7.42 (m, 6H), 7.01 (d, J=8.9 Hz, 1H), 5.19 (s, 2H).

### EXAMPLE 2

### Formic acid, 2-benzyloxy-5-chloro-phenyl ester

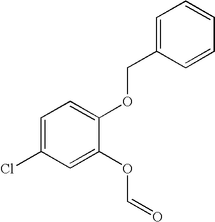

In a 1 L one-neck flask (equipped with magnetic stirrer, and nitrogen inlet, was charged 2-benzyloxy-5-chloro-benzaldehyde (8, 28.1 g, 0.114 mol) and dichloromethane (360 mL). To this solution was added m-CPBA (˜75%, 31.4 g, 0.137 mol). The reaction mixture was stirred at room temperature for 17 h (overnight) to yield a white slurry. The white solid was removed by filtration, then the filtrate was washed with 10% (w/w) sodium bisufite (200 mL) and each phase was tested by starch iodide paper to see if any oxidant remained. This test was negative and the organic phase was washed with saturated sodium bicarbonate (2×150 mL), dried (MgSO₄), and concentrated to yield crude product, as a solid. The crude product was used in subsequent steps without further purification.

¹H NMR (CDCl₃) δ ppm: 8.25 (s, 1H), 7.37 (m, 5H), 7.15 (m, 2H), 6.95 (d, J=8.7 Hz, 1H), 5.09 (s, 2H).

### EXAMPLE 3

### 2-Benzyloxy-5-chloro-phenol

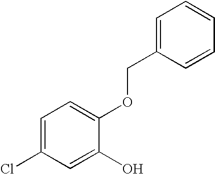

A one-neck flask (equipped with magnetic stirring, and a nitrogen inlet), was charged with crude formic acid, 2-benzyloxy-5-chloro-phenyl ester (9, 28 g, 0.107 mol), sodium methoxide (25% (w/w) in methanol, 26.5 mL, 0.123 mol), and methanol (175 mL). The reaction mixture was stirred for 18 h (overnight). The reaction mixture was concentrated to a red oil that was partitioned between saturated ammonium chloride (200 mL) and ethyl ether (200 mL). The aqueous was extracted with ethyl ether (100 mL) and the combined organics were washed with brine (100 mL), dried (MgSO₄), and concentrated to yield the title compound as a oil. The product was used in subsequent steps without further purification.

¹H NMR (CDCl₃) δ ppm: 7.41 (m, 5H), 6.95 (d, J=1.8 Hz, 1H), 6.82 (m, 2H), 5.71 (s, 1H), 5.09 (s, 2H).

### EXAMPLE 4

### (R)-2-(2-Benzyloxy-5-chloro-phenoxymethyl)-oxirane

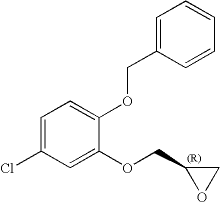

In a 500 mL, three-necked flask (equipped with a magnetic stir bar, thermocouple, and a nitrogen inlet) was charged 2-benzyloxy-5-chloro-phenol (10, 11.3 g, 48.2 mmol), (R)-glycidyl m-nitrophenylsulfonate (11.3 g, 43.8 mmol), and N,N-dimethylformamide (200 mL). The reaction mixture was heated to 40° C. for 12 hrs, then cooled to room temperature and poured into water (300 mL). The aqueous phase was extracted with ethyl ether (3×200 mL). The combined organic phases were washed with 1M sodium hydroxide (aq), brine (3×100 mL), dried (MgSO₄), and concentrated in vacuo to yield crude product as an oil. The oil was dissolved in 1:1 heptane-dichloromethane and loaded onto an Isco cartridge (120 g silica gel) and eluted to yield the title compound as an oil.

¹H NMR (CDCl₃) δ ppm: 7.38 (m, 5H), 6.94 (d, J=2.5 Hz, 1H), 6.85 (m, 2H), 5.11 (s, 2H), 4.28 (dd, J=11.4, 3.3 Hz, 1H), 4.01 (dd, J=11.3, 5.5 Hz, 1H), 3.38 (m, 1H), 2.89 (dd, J=5.1, 4.9 Hz, 1H), 2.77 (dd, J=5.0, 2.6 Hz, 1H).

### EXAMPLE 5

### (R)-4-Chloro-2-oxiranylmethoxy-phenol

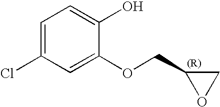

A Parr reaction bottle was charged with 5% palladium on carbon (dry, 1.0 g), (R)-2-(2-benzyloxy-5-chloro-phenoxymethyl)-oxirane (11, 11.7 g, 40.2 mmol), and ethyl acetate (250 mL). The reaction mixture was agitated on a Parr shaker at room temperature under 5-7 psi of hydrogen gas for 2.5 h. The catalyst was removed by filtration and the ethyl acetate removed in vacuo to yield the title compound as an oil that solidified upon standing. The product was used in subsequent steps without further purification.

¹H NMR (CDCl₃) δ ppm: 6.87 (m, 3H), 6.09 (bs, 1H), 4.25 (dd, J=11.4, 2.6 Hz, 1H), 3.98 (dd, J=11.3, 5.7 Hz, 1H), 3.39 (m, 1H), 2.96 (t, J=4.6 Hz, 1H), 2.85 (dd, J=5.2, 2.7 Hz, 1H).

### EXAMPLE 6

### (S)-(6-Chloro-2,3-dihydro-benzo[1,4]dioxin-2-yl)-methanol

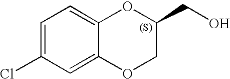

A one-neck flask (equipped with magnetic stirring, and a nitrogen inlet), was charged (R)-4-chloro-2-oxiranylmethoxy-phenol (12, 8.1 g, 40.4 mmol), sodium methoxide (25% (w/w) in methanol, 10.5 mL, 48.4 mmol), and methanol (90 mL). The reaction was stirred for 18 h (overnight). The reaction mixture was concentrated to a red oil that was partitioned between water (250 mL) and ethyl ether (200 mL). The aqueous layer was extracted with ethyl ether (2×100 mL), and the combined organics were washed with 1M aqueous sodium hydroxide (150 mL), brine (2×100 mL), dried (MgSO₄), and concentrated to yield crude product. The crude product was dissolved in dichloromethane and loaded onto a Biotage 40M (90 g silica gel) and eluted with dichloromethane (250 mL) and 1:19 ethyl acetate-dichloromethane (1 L) to yield the title compound as a white solid (19415-132A).

Melting Point: 73-75° C.

Optical rotation: [α]_{D}=−58.0° (c 1.99, MeOH, 23° C.)

¹H NMR (CDCl₃) δ ppm: 6.82 (m, 3H), 4.30 (dd, J=11.1, 2.2 Hz, 1H), 4.24 (m, 1H), 4.10 (dd, J=11.2, 7.4 Hz, 1H), 3.87 (m, 2H), 1.95 (t, J=6.6 Hz, 1H).

### EXAMPLE 7

### (R)-1-(4-Chloro-2-oxiranylmethoxy-phenyl)-ethanone

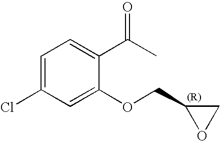

In a 22 L, four-necked flask (equipped with a mechanical stirrer, thermocouple, Teflon stopper, and a argon inlet) was charged 4-chloro-2-hydroxy-acetophenone (13, 687.2 g, 4.03 mol) and N,N-dimethylformamide (14.0 L). To this solution was added potassium carbonate (613 g, 4.43 mol) and (R)-glycidyl m-nitrophenylsulfonate (1.045 kg, 4.03 mol). The reaction mixture was heated to 40° C. for 20 h (overnight) then cooled to room temperature and split into two portions. Each portion was poured into water (9 L) and extracted with methyl tert-butyl ether (3×2 L) and ethyl ether (2 L). The two organic phases were each washed with brine (2×4 L), dried (MgSO₄), and concentrated in vacuo to yield crude product, which was used in subsequent steps without further purification.

¹H NMR (CDCl₃) δ ppm: 7.71 (d, J=8.5 Hz, 1H), 7.02 (dd, J=8.8, 1.7 Hz, 1H), 6.95 (d, J=1.8 Hz, 1H), 4.39 (dd, J=11.0, 2.8 Hz, 1H), 3.98 (dd, J=10.8, 6.1 Hz, 1H), 3.41 (m, 1H), 2.96 (t, J=4.4 Hz, 1H), 2.78 (dd, J=5.1, 2.7 Hz, 1H), 2.64 (s, 3H).

A small portion of the product prepared as described above was chromatographed (Isco silica—40 g) to yield a white powder:

MP: 79-80° C.

Optical Rotation: [α]_{D}=−19.4° (c 2.02, MeOH, 23° C.).

High-Resolution MS: (as C₁₁H₁₂O₃Cl, M^{+H}): Calculated: 227.48; Measured: 227.05

### EXAMPLE 8

### (R)-Acetic acid, 4-chloro-2-oxiranylmethoxy-phenyl ester

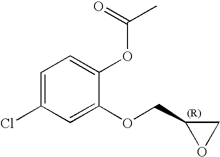

In a 22 L four-necked flask (equipped with mechanical stirrer, two stoppers, and a condenser with a argon inlet), was charged (R)-1-(4-chloro-2-oxiranylmethoxy-phenyl)-ethanone (14, 815 g, 3.60 mol) and dichloromethane (8.8 L). To this solution was added m-CPBA (˜75% Lancaster, 993 g, 4.31 mol oxidant). The reaction mixture was heated at mild reflux for 22 h over 3 days (heat was only applied with supervision). We then added additional m-CPBA (100 g, ˜0.36 mol) and continued reflux for 8 h. The reaction mixture was then allowed to cool overnight to yield a white slurry upon cooling. The white solid was removed by filtration and the filtrate was split into four portions. Each portion was washed with 10% (w/w) sodium bisufite (1 L) and both phases were tested by starch iodide paper to see if any oxidant remained. This test was negative and the aqueous phase was back extracted with dichloromethane (2×250 mL) and the combined organic phases (four portions) were washed with saturated sodium bicarbonate (3×500 mL), dried (MgSO₄), and concentrated to yield crude product, which was used in subsequent steps without further purification.

¹H NMR (CDCl₃) δ ppm: 6.96 (m, 3H), 4.25 (dd, J=10.9, 2.4 Hz, 1H), 3.94 (dd, J=11.5, 5.6 Hz, 1H), 3.30 (m, 1H), 2.88 (t, J=4.8 Hz, 1H), 2.71 (dd, J=4.8, 2.6 Hz, 1H), 2.31 (s, 3H).

A small portion of the crude product prepared as described above was chromatographed (Isco silica—12 g) to yield a clear oil:

Optical Rotation: [α]_{D}=−8.29° (c 2.05, MeOH, 23° C.).

High-Resolution MS: (as C₁₁H₁₁O₃Cl, M⁺): Calculated: 242.03; Measured: 242.03

### EXAMPLE 9

### (S)-(6-Chloro-2,3-dihydro-benzo[1,4]dioxin-2-yl)-methanol

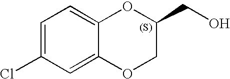

A four-necked 12 L flask (equipped with mechanical stirring, two stoppers, and a nitrogen inlet), was charged (R)-acetic acid, 4-chloro-2-oxiranylmethoxy-phenyl ester (15, 1.019 kg impure, assumed 874 g, 3.60 mol), sodium methoxide (25% (w/w) in methanol, 972 mL, 4.50 mol), and methanol (6.0 L). The reaction mixture was stirred for 20 h (overnight) and then treated with 3 M aqueous sodium hydroxide (500 mL) and stirred for an additional 6 days. The reaction mixture was concentrated to a red oil that was diluted with water to a volume of 4.0 L. This material was split into two portions and each aqueous phase was extracted with methyl tert-butyl ether (2×1.0 L) and ethyl ether (1 L). The organic phases were washed with 10% aqueous sodium bisulfite (500 mL), saturated sodium bicarbonate (500 mL), brine (2×500 mL), dried (MgSO₄), and concentrated to yield crude product. The crude product was used in subsequent steps without further purification.

¹H NMR (CDCl₃) δ ppm: 6.89 (m, 1H), 6.2 (m, 2H), 4.30 (dd, J=11.3, 2.2 Hz, 1H), 4.23 (m, 1H), 4.10 (dd, J=10.9, 7.6 Hz, 1H), 3.91 (dd, J=12.0, 4.2 Hz, 1H), 3.83 (dd, J=12.1, 5.4 Hz, 1H).

A small portion of the crude product prepared as described above was chromatographed (Isco silica—12 g) to yield a white solid:

Melting Point: 79-81° C.

Optical rotation: [α]_{D}=−58.2° (c 2.05, MeOH, 23° C.);

Chiral HPLC: Chiralpak AS-H, Hexane/IPA (97:3), Rₜ=16.095 min, >99% ee.

High-Resolution MS: (as C₉H₉O₃Cl, M⁺): Calculated: 200.02: Measured: 200.02

### EXAMPLE 10

### (R)-Toluene-4-sulfonic acid, 6-chloro-2,3-dihydro-benzo[1,4]dioxin-2-yl-methyl ester

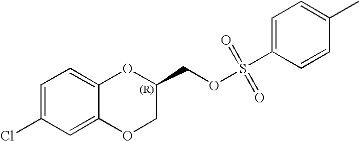

To a 12 L, four necked flask (equipped with mechanical stirrer, nitrogen inlet, and two stoppers) was charged (S)-(6-chloro-2,3-dihydro-benzo[1,4]dioxin-2-yl)-methanol (2, 300 g, 1.50 mol) and 4.5 L of tetrahydrofuran/dichloromethane (9:1). This solution was treated with N-methylmorpholine (362 mL, 3.29 mol), N,N-dimethylaminopyridine (36.4 g, 0.298 mol), and p-toluenesulfonyl chloride (341 g, 1.79 mol). The reaction mixture was stirred under nitrogen at room temperature for 10 days (reaction appeared complete after ˜4 days). The reaction mixture was filtered to remove amine hydrochloride and split into two portions. Each portion was stirred with 20% aqueous potassium bicarbonate (3 L) for 2.5 h. Each portion was then extracted with i-propyl acetate (2×1.5 L) and the organics were washed with 2 M hydrochloric acid (2×1 L), saturated sodium bicarbonate (2×1 L), brine (500 mL), and dried (MgSO₄). The resulting mixture was concentrated to yield crude product. The crude product was dissolved in dichloromethane/heptane (1:3) and loaded onto a Biotage 150L (5 kg silica gel) and eluted (30 psi) with heptane (8 L), 1:9 ethyl acetate-heptane (24 L), 15:85 ethyl acetate-heptane (48 L), and 1:2 ethyl acetate-heptane (18 L) to yield the title compound as an off-white solid.

¹H NMR (DMSO-d6) δ ppm: 7.79 (d, J=7.9 Hz, 2H), 7.47 (d, J=8.0 Hz, 2H), 6.94 (d, J=2.5 Hz, 1H), 6.87 (dd, J=8.8, 2.5 Hz, 1H), 6.8 (d, J=8.8 Hz, 1H), 4.46 (m, 1H), 4.35 (dd, J=11.7, 3.1 Hz, 1H), 4.29 (dd, J=11.7, 2.5 Hz, 1H), 4.20 (dd, J=11.3, 6.4 Hz, 1H), 3.99 (dd, J=11.5, 6.2 Hz, 1H), 2.42 (s, 1H).

Optical rotation: [α]_{D}=−34.73° (c 2.02, MeOH, 23° C.).

### EXAMPLE 11

### (S)-2-(6-Chloro-2,3-dihydro-benzo[1,4]dioxin-2-ylmethyl)-isoindole-1,3-dione

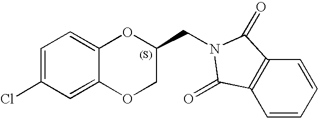

A 12-L four necked flask (equipped with mechanical stirrer, air condenser topped with a nitrogen inlet, and two stoppers) was charged with (R)-toluene-4-sulfonic acid, 6-chloro-2,3-dihydro-benzo[1,4]dioxin-2-yl-methyl ester (3, 443 g, 1.25 mol), potassium phthalimide (301 g, 1.62 mol), and N,N-dimethylformamide (5 L). The reaction mixture was heated to 100° C. for 2 h then cooled to room temperature over 16 h (overnight). The reaction mixture was diluted into water (6 L) and stirred for 2.5 h. The resulting white solid was collected by vacuum filtration and dried in a vacuum oven at 55° C. to yield the title compound as an off-white solid.

¹H NMR (DMSO-d6) δ ppm: 7.84 (m, 4H), 6.98 (d, J=2.9 Hz, 1H), 6.87 (dd, J=8.4, 2.8 Hz, 1H), 6.83 (d, J=8.3 Hz, 1H), 4.49 (qd, J=6.1, 2.2, 1H), 4.36 (dd, J=11.4, 2.7 Hz, 1H), 4.13 (dd, J=11.9, 2.5 Hz, 1H), 3.88 (m, 2H).

Melting Point: 126-127° C.

### EXAMPLE 12

### (S)-(6-Chloro-2,3-dihydro-benzo[1,4]dioxin-2-yl)-methylamine

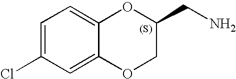

A 12-L four necked flask (equipped with mechanical stirrer, water condenser topped with a nitrogen inlet, and two stoppers) was charged with (S)-2-(6-chloro-2,3-dihydro-benzo[1,4]dioxin-2-ylmethyl)-isoindole-1,3-dione (4, 370 g, 1.12 mol), hydrazine (65.2 mL, 2.08 mol), and ethanol (4 L). The reaction mixture was heated to reflux for 1.5 h and then cooled to room temperature. The reaction mixture was acidified with 1 M hydrochloric acid (2 L) and filtered. The filtrate was concentrated to remove ethanol then basified with 3 M sodium hydroxide (1 L) and extracted with ethyl ether (2 L, then 2×1 L). The combined organics were washed with brine (2×600 mL), dried (MgSO₄), and concentrated to yield the title compound as a white solid, which was used in subsequent steps without further purification.

¹H NMR (DMSO-d6) δ ppm: 6.95 (d, J=1.9 Hz, 1H), 6.87 (m, 2H), 4.37 (dd, J=11.5, 2.6, 1H), 4.07 (m, 1H), 3.98 (dd, J=10.9, 7.7 Hz, 1H), 2.83 (dd, J=12.7, 5.4, 1H), 2.77 (dd, J=12.7, 6.1, 1H).

Melting Point: 70-71° C.

Optical rotation: [α]_{D}=−81.0° (c 2.00, MeOH, 23° C.).

### EXAMPLE 13

### (S)—N-(6-Chloro-2,3-dihydro-benzo[1,4]dioxin-2-ylmethyl)-sulfamide

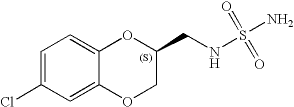

A 12-L four necked flask (equipped with mechanical stirrer, water condenser topped with a nitrogen inlet, and two stoppers) was charged with (S)-(6-chloro-2,3-dihydro-benzo[1,4]dioxin-2-yl)-methylamine (5, 214 g, 1.07 mol), sulfamide (412 g, 4.29 mol), and iso-propyl acetate (4 L). The reaction mixture was then heated to reflux for a total of 15 h over 3 days and then cooled to room temperature. The reaction mixture was chilled in an ice-bath and the residues were collected by filtration and washed with iso-propyl acetate. The filtrate was washed with 1 M hydrochloric acid (3 L), dried (MgSO₄), and concentrated to yield crude product. The entire portion of crude product was dissolved in ethyl acetate and absorbed onto silica gel (600 g) and loaded into a Biotage sample induction module, then eluted onto a Biotage 150M (2.5 g silica gel) using heptane (2 L), 1:9 ethyl acetate-heptane (4 L), 3:7 ethyl acetate-heptane (12 L), and 1:1 ethyl acetate-heptane (16 L) to yield the product along with mixed fractions. The mixed fractions were re-chromatographed on a Biotage 75L (800 g silica) using heptane (1 L), 1:9 ethyl acetate-heptane (2 L), 3:7 ethyl acetate-heptane (6 L), and 1:1 ethyl acetate-heptane (8 L) to yield additional product. The two lots of product were combined to yield the title compound as an off-white solid.

The title compound prepared according to the procedure as described in this Example yielded form (I-SA).

¹H NMR (DMSO-d6) δ ppm: 6.98 (d, J=1.9 Hz, 1H), 6.89 (m, 3H), 6.67 (bs, 2H), 4.36 (dd, J=11.7, 1.6, 1H), 4.28 (m, 1H), 4.00 (dd, J=11.5, 6.8 Hz, 1H), 3.19 (m, 1H), 3.11 (m, 1H).

Melting Point: 99-100° C.

Optical rotation: [α]_{D}=−57.6° (c 2.14, MeOH, 23° C.).

Chiral HPLC: Chiralpak AD-H, Hex(0.1% TEA)/IPA (80:20), Rₜ=11.407 min, >99% ee.

Elemental Analysis for C₉H₁₁ClN₂O₄S:

Calculated: % C 38.78, % H 3.98, % Cl 12.72, % N 10.05, % S 11.51.

Measured: % C 38.81, % H 3.74, % Cl 12.83, % N 9.93, % S 11.53.

### EXAMPLE 14

### Water Recrystallization of (S)—N-(6-Chloro-2,3-dihydro-benzo[1,4]dioxin-2-ylmethyl)-sulfamide

Distilled water (5 mL) was added to (S)—N-(6-Chloro-2,3-dihydro-benzo[1,4]dioxin-2-ylmethyl)-sulfamide (0.050 g) in a test tube and the resulting mixture was heated. The white solid was observed to dissolve in the water before the water boiled. As the water cooled, material dropped out as a suspension and formed white needle-like crystals over two hours. The crystals were filtered off, rinsed with water, air dried, and collected to yield crystalline form (I-SB) as herein defined.

### EXAMPLE 15

### (R)-Toluene-4-sulfonic acid, (7-chloro-2,3-dihydro-benzo[1,4]dioxin-2-yl)-methyl ester

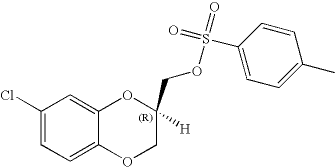

(S)-(7-Chloro-2,3-dihydro-1,4-benzodioxin-2-yl)methanol {[α]_{D}=−35.6° (c=1.45, EtOH), 7.12 g, 35.4 mmol}, prepared according to the method as disclosed in A. M. Birch et al. Journal of Medicinal Chemistry 1999, 42, 3342-3355, was dissolved in pyridine (50 mL) and cooled to 0° C. To the resulting mixture was added p-toluenesulfonyl chloride (6.68 g, 35.5 mmol) and the reaction mixture stirred at room temperature for 20 h. The reaction mixture was then cooled in an ice bath and 1N HCl (750 mL) was added. The reaction mixture was extracted with diethyl ether (3×, 200 mL). The combined diethyl ether was washed with 1N HCl (2×, 250 mL), water, brine (2×) and dried (MgSO₄) and evaporated in vacuo to yield the title compound as a white solid.

¹H NMR (CDCl₃) δ 7.79 (d, J=8.4 Hz, 2H), 7.37 (d, J=8.5 Hz, 2H), 6.76 (m, 3H), 4.40 (m, 1H), 4.21 (m, 3H), 4.03 (dd, J=6.2, 11.6 Hz, 1H), 2.61 (s, 3H).

### EXAMPLE 16

### (S)-2-(7-Chloro-2,3-dihydro-benzo[1,4]dioxin-2-ylmethyl)-isoindole-1,3-dione

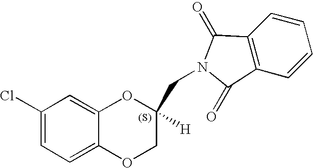

(R)-Toluene-4-sulfonic acid, (7-chloro-2,3-dihydro-benzo[1,4]dioxin-2-yl)-methyl ester (10.65 g), prepared as in Example 15 above) was combined with potassium phthalimide (8.90 g, 48 mmol) in DMF (100 mL) and the resulting mixture was heated to reflux for 1 h, then cooled to room temperature and poured into vigorously stirring ice water (750 mL) and stirred 30 min. The resulting white solid was filtered and washed several times with water, then dried under vacuum (16 h) to yield the title compound as white powdery solid.

¹H NMR (CDCl₃) δ 7.89 (m, 2H), 7.75 (m, 2H), 6.82 (m, 3H), 4.50 (m, 1H), 4.29 (dd, J=2.3, 11.6 Hz, 1H), 4.05 (m, 2H), 3.89 (dd, J=5.4, 14.2 Hz, 1H).

### EXAMPLE 17

### (S)-(7-Chloro-2,3-dihydro-benzo[1,4]dioxin-2-yl)-methylamine

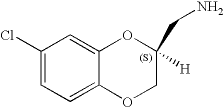

(S)-2-(7-chloro-2,3-Dihydro-benzo[1,4]dioxin-2-ylmethyl)-isoindole-1,3-dione (8.1 g, 24.6 mmol; prepared as in Example 16 above) was combined with hydrazine (1.57 g, 49 mmol) in EtOH (120 mL) and heated at reflux for 2 h, then cooled to room temperature. To the reaction mixture was then added 1N HCl to pH 2.0 and the resulting mixture was stirred for 45 min. The resulting white solid was filtered and washed several times with fresh EtOH and the solid discarded. The filtrate was evaporated in vacuo to yield a solid, which was partitioned between diethyl ether and dilute aqueous NaOH. The diethyl ether solution was washed once with brine, dried (Na₂SO₄) and evaporated in vacuo to yield the title compound as a clear viscous oil.

MS 200 (MH⁺)

¹H NMR (CDCl₃) δ 6.90 (dd, J=1.54, 1.1 Hz, 1H), 6.79 (m, 2H), 4.26 (dd, J=11.2, 2.2 Hz, 1H), 4.13 (m, 1H), 3.98 (dd, J=11.3, 7.5 Hz, 1H), 2.98 (m, 2H), 1.25 (bd s, NH₂)

Optical Rotation [α]_{D}=−54.2° (c=1.55, CHCl₃)

### EXAMPLE 18

### (S)—N-(7-Chloro-2,3-dihydro-benzo[1,4]dioxin-2-ylmethyl)-sulfamide (Compound of Formula (I-S))

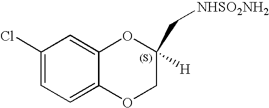

(S)-(7-chloro-2,3-dihydro-benzo[1,4]dioxin-2-yl)-methylamine (4.7 g, 23.5 mmol) and sulfamide (4.51 g, 47 mmol) were refluxed in dioxane (125 mL) for 3 h, then cooled to room temperature, filtered and evaporated in vacuo to yield a solid. The solid (crude product) was purified by flash column chromatography (DCM:MeOH 20:1) to yield a white solid, which was recrystallized from ethyl acetate/hexane to yield the title compound as a white crystalline solid.

mp 118-119° C.

MS 277 (M-1)

Optical Rotation [α]_{D}=−40.0° (c=1.20, MeOH)

¹H NMR (DMSOd6) δ6.97 (d, J=2.2 Hz, 1H), 6.90 (m, 3H), 6.67 (s, 2H, NH₂), 4.35 (m, 2H), 4.01 (m, 1 H), 3.15 (m, 2 H)

Chemical Analysis:

Calculated C, 38.78; H, 3.98; N, 10.05; S, 11.51

Measured: C, 38.83; H, 3.88; N, 10.08; S, 11.31

### EXAMPLE 19

### Liquid Formulation

The compound of formula (I-S), prepared for example as described above, was formulated according to known methods into liquid formulations of 25 mg and 100 mg, respectively, with components as listed in Table 4 below.

TABLE 4
Liquid Formulations

| | | 25 mg/mL | | 100 mg/mL | |
|---|---|---|---|---|---|
| Component | Role | Suspension | | Suspension | |
| Compound of formula | Active | 25 | mg | 100 | mg |
| (I-S) | | | | | |
| Hypromellose | Suspending | 5 | mg | 5 | mg |
| (also known as HPMC or | agent | | | | |
| hydroxypropylmethyl- | | | | | |
| cellulose) | | | | | |
| Purified water | Solvent | q.s. ad. 1 | mL | q.s. ad. 1 | mL |

### EXAMPLE 20

### Prophetic Example

As a specific embodiment of an oral composition, 100 mg of the compound prepared as in Example 18 is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A process for the preparation of a compound of formula (V) 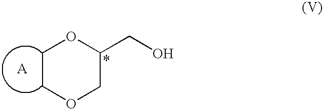
wherein 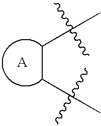
is selected from the group consisting of 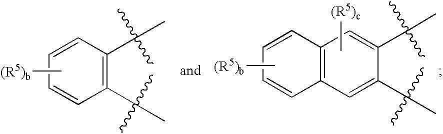
b is an integer from 0 to 4; and wherein c is an integer from 0 to 2;
each R⁵ is independently selected from the group consisting of halogen, lower alkyl and nitro;
comprising 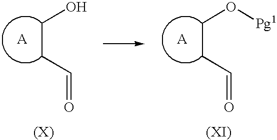
protecting a compound of formula (X); to yield the corresponding compound of formula (XI), wherein Pg¹ is an alcohol protecting group; 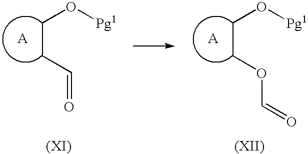
reacting the compound of formula (XI) with an oxidizing agent; in an organic solvent; to yield the corresponding compound of formula (XII); 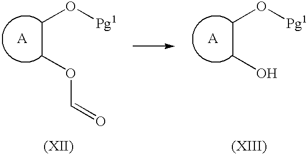
reacting the compound of formula (XII) with an organic or inorganic base; in an organic solvent, in a mixture of organic solvents or in a mixture of one or more organic solvents and water; to yield the corresponding compound of formula (XIII); 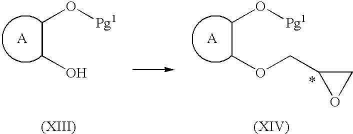
reacting the compound of formula (XIII) with a source of epoxy-methylene; in the presence of an inorganic base; at a temperature greater than about room temperature; in an organic solvent; to yield the corresponding compound of formula (XIV); 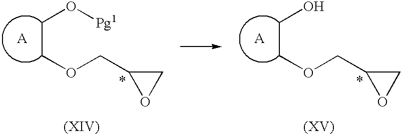
de-protecting the compound of formula (XIV); to yield the corresponding compound of formula (XV); 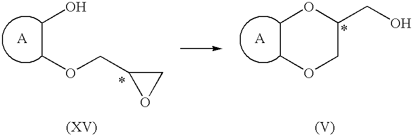
reacting the compound of formula (XV) with an organic or inorganic base; in a organic solvent; to yield the corresponding compound of formula (V).

2. A process as in claim 1, wherein Pg¹ is selected from the group consisting of benzyl, allyl, 2-(trimethylsilyl)ethoxymethyl, t-butyl-diphenylsilyl, 4-nitro-benzyl, 4-methoxybenzyl, methoxymethyl and ethoxyethyl.

3. A process as in claim 2, wherein Pg¹ is selected from the group consisting of benzyl and allyl.

4. A process as in claim 1, wherein the oxidizing agent is m-CPBA.

5. A process as in claim 1, wherein the organic or inorganic base reacted with compound of formula (XII) is NaOCH₃.

6. A process as in claim 1, wherein the source of epoxy-methylene is selected form the group consisting of glycidyl-m-nosylate and glycidyl-tosylate.

7. A process as in claim 1, wherein the source of epoxy-methylene is selected from the group consisting of (R)-glycidyl-m-nosylate and (R)-glycidyl tosylate.

8. A process as in claim 3, wherein the compound of formula (XIV) is de-protected by reacting the compound of formula (XIV) with hydrogen or a source of hydrogen.

9. A process as in claim 1, wherein the organic or inorganic base reacted with the compound of formula (V) is NaOCH₃.

10. A process as in claim 1, wherein

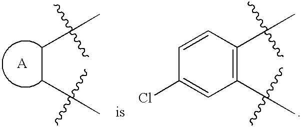

11. A process for the preparation of compounds of formula (I) 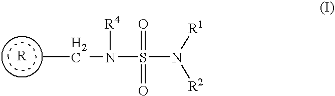
wherein 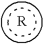
is selected from the group consisting of 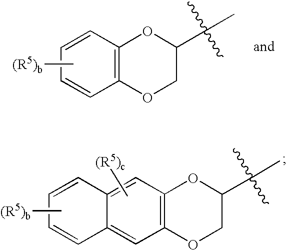
b is an integer from 0 to 4; and wherein c is an integer from 0 to 2;
each R⁵ is independently selected from the group consisting of halogen, lower alkyl and nitro;
R⁴ is selected from the group consisting of hydrogen and lower alkyl;
R¹ and R² are each independently selected from the group consisting of hydrogen and lower alkyl;
or pharmaceutically acceptable salts thereof;
comprising 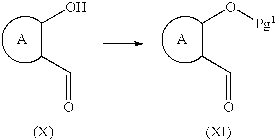
protecting a compound of formula (X) wherein 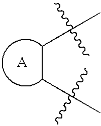
is selected from the group consisting of 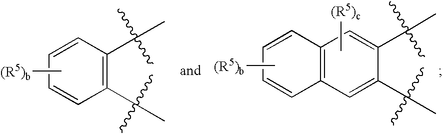
to yield the corresponding compound of formula (XI), wherein Pg¹ is an alcohol protecting group; 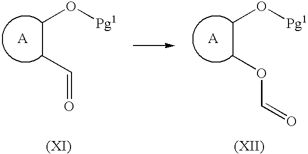
reacting the compound of formula (XI) with an oxidizing agent; in an organic solvent; to yield the corresponding compound of formula (XII); 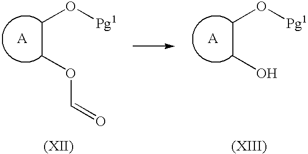
reacting the compound of formula (XII) with an organic or inorganic base; in an organic solvent, in a mixture of organic solvents or in a mixture of one or more organic solvents and water; to yield the corresponding compound of formula (XIII); 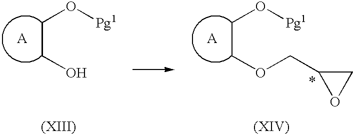
reacting the compound of formula (XIII) with a source of epoxy-methylene; in the presence of an inorganic base; at a temperature greater than about room temperature; in an organic solvent; to yield the corresponding compound of formula (XIV); 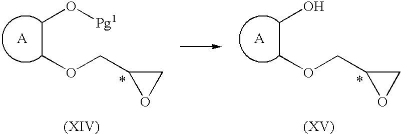
de-protecting the compound of formula (XIV); to yield the corresponding compound of formula (XV); 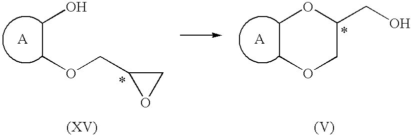
reacting the compound of formula (XV) with an organic or inorganic base; in a organic solvent; to yield the corresponding compound of formula (V). 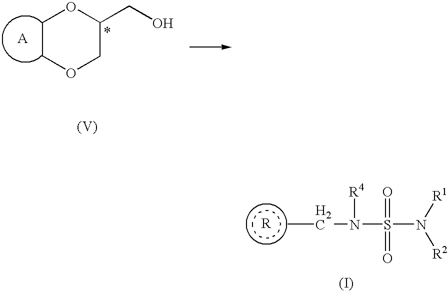
reacting the compound of formula (V); to yield the corresponding compound of formula (I).

12. A process as in claim 11, wherein PG¹ is selected from the group consisting of benzyl, allyl, 2-(trimethylsilyl)ethoxymethyl, t-butyl-diphenylsilyl, 4-nitro-benzyl, 4-methoxybenzyl, methoxymethyl and ethoxyethyl.

13. A process as in claim 12, wherein Pg¹ is selected from the group consisting of benzyl and allyl.

14. A process as in claim 11, wherein the oxidizing agent is m-CPBA.

15. A process as in claim 11, wherein the organic or inorganic base reacted with compound of formula (XII) is NaOCH₃.

16. A process as in claim 11, wherein the source of epoxy-methylene is selected from the group consisting of glycidyl-m-nosylate and glycidyl-tosylate.

17. A process as in claim 11, wherein the source of epoxy-methylene is selected from the group consisting of (R)-glycidyl-m-nosylate and (R)-glycidyl tosylate.

18. A process as in claim 13, wherein the compound of formula (XIV) is de-protected by reacting the compound of formula (XIV) with hydrogen or a source of hydrogen.

19. A process as in claim 11, wherein the organic or inorganic base reacted with the compound of formula (V) is NaOCH₃.

20. A process as in claim 1, wherein 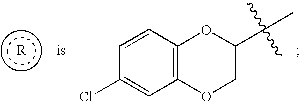
R¹ is hydrogen, R² is hydrogen and R⁴ is hydrogen.

21. A process for the preparation of a compound of formula (V-S) 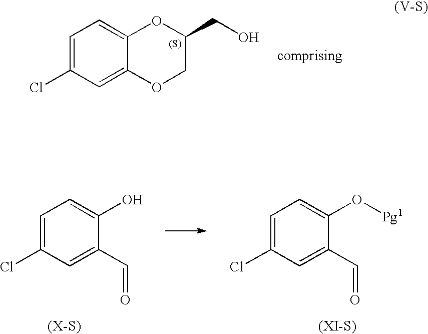
protecting a compound of formula (X-S); to yield the corresponding compound of formula (XI-S), wherein Pg¹ is an alcohol protecting group; 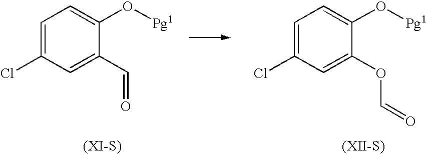
reacting the compound of formula (XI-S) with an oxidizing agent; in an organic solvent; to yield the corresponding compound of formula (XII-S); 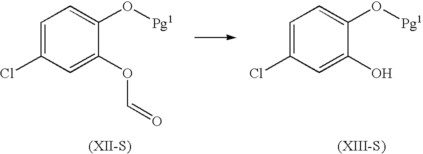
reacting the compound of formula (XII-S) with an organic or inorganic base; in an organic solvent, in a mixture of organic solvents or in a mixture of one or more organic solvents and water; to yield the corresponding compound of formula (XIII-S); 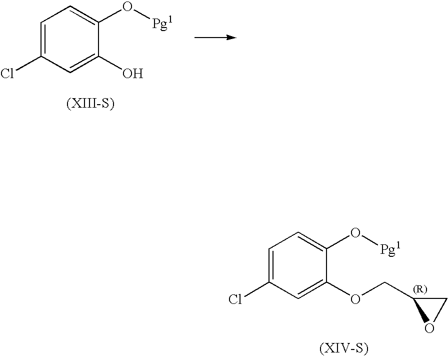
reacting the compound of formula (XIII-S) with a source of (R)-epoxy-methylene; in the presence of an inorganic base; at a temperature greater than about room temperature; in an organic solvent; to yield the corresponding compound of formula (XIV-S); 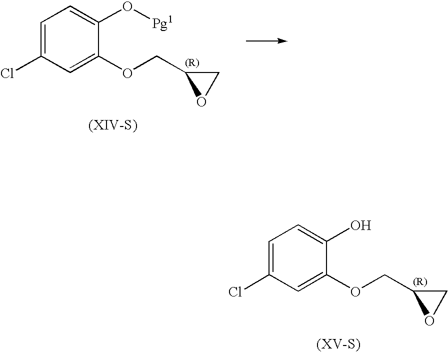
de-protecting the compound of formula (XIV-S); to yield the corresponding compound of formula (XV-S); 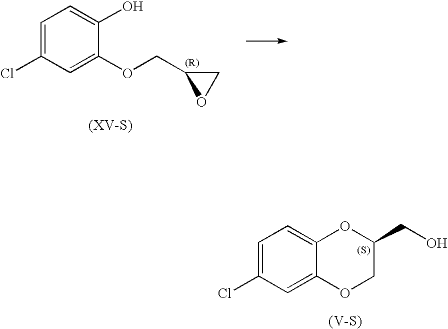
reacting the compound of formula (XV-S) with an organic or inorganic base; in a organic solvent; to yield the corresponding compound of formula (V-S).

22. A process as in claim 21, wherein Pg¹ is selected from the group consisting of benzyl, allyl, 2-(trimethylsilyl)ethoxymethyl, t-butyl-diphenylsilyl, 4-nitro-benzyl, 4-methoxybenzyl, methoxymethyl and ethoxyethyl.

23. A process as in claim 22, wherein Pg¹ is selected from the group consisting of benzyl and allyl.

24. A process as in claim 21, wherein the oxidizing agent is m-CPBA.

25. A process as in claim 21, wherein the organic or inorganic base reacted with compound of formula (XII-S) is NaOCH₃.

26. A process as in claim 21, wherein the source of (R)-epoxy-methylene is selected from the group consisting of (R)-glycidyl-m-nosylate and (R)-glycidyl tosylate.

27. A process as in claim 23, wherein the compound of formula (XIV-S) is de-protected by reacting the compound of formula (XIV) with hydrogen or a source of hydrogen.

28. A process as in claim 21, wherein the organic or inorganic base reacted with the compound of formula (V-S) is NaOCH₃.

29. A process for the preparation of a compound of formula (I-S) comprising the process of claim 21; and further comprising 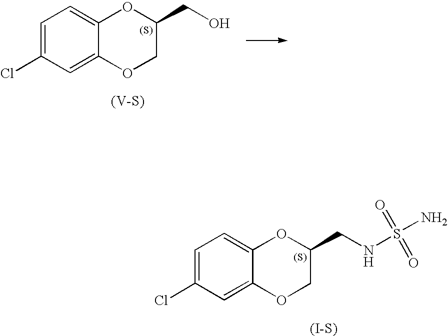
reacting the compound of formula (V-S); to yield the corresponding compound of formula (I-S).

30. A process for the preparation of a compound of formula (I-S) 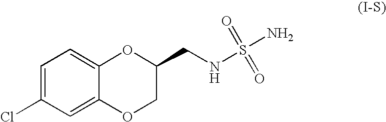
or a pharmaceutically acceptable salt thereof comprising 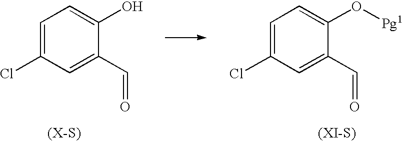
protecting a compound of formula (X-S); to yield the corresponding compound of formula (XI-S), wherein Pg¹ is an alcohol protecting group; 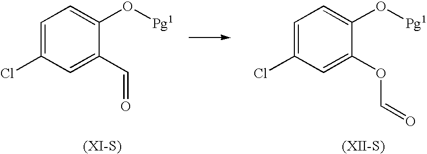
reacting the compound of formula (XI-S) with an oxidizing agent; in an organic solvent; to yield the corresponding compound of formula (XII-S); 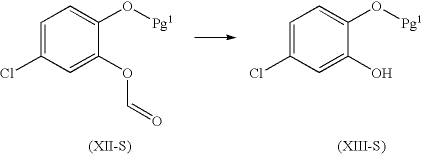
reacting the compound of formula (XII-S) with an organic or inorganic base; in an organic solvent, in a mixture of organic solvents or in a mixture of one or more organic solvents and water; to yield the corresponding compound of formula (XIII-S); 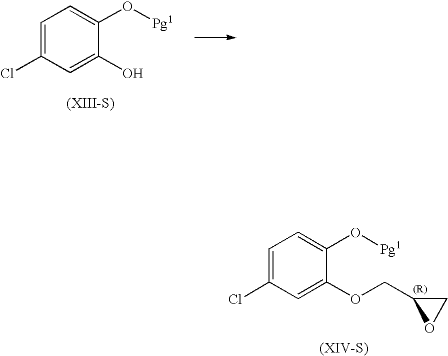
reacting the compound of formula (XIII-S) with a source of (R)-epoxy-methylene; in the presence of an inorganic base; at a temperature greater than about room temperature; in an organic solvent; to yield the corresponding compound of formula (XIV-S); 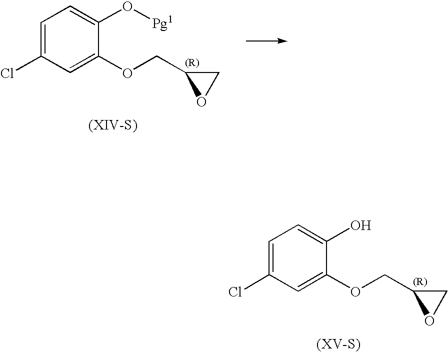
de-protecting the compound of formula (XIV-S); to yield the corresponding compound of formula (XV-S); 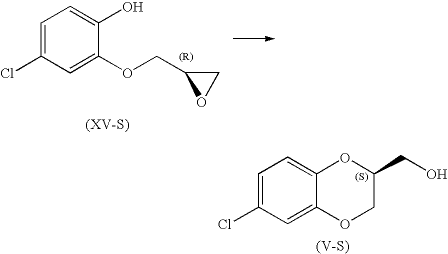
reacting the compound of formula (XV-S) with an organic or inorganic base; in a organic solvent; to yield the corresponding compound of formula (V-S); 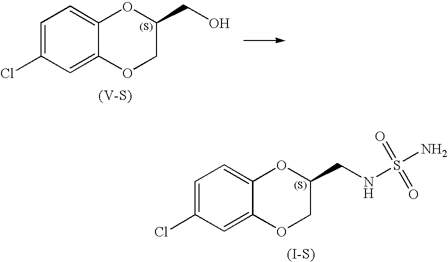
reacting the compound of formula (V-S); to yield the corresponding compound of formula (I-S).

31. A process as in claim 30, wherein Pg¹ is selected from the group consisting of benzyl, allyl, 2-(trimethylsilyl)ethoxymethyl, t-butyl-diphenylsilyl, 4-nitro-benzyl, 4-methoxybenzyl, methoxymethyl and ethoxyethyl.

32. A process as in claim 31, wherein Pg¹ is selected from the group consisting of benzyl and allyl.

33. A process as in claim 30, wherein the oxidizing agent is m-CPBA.

34. A process as in claim 30, wherein the organic or inorganic base reacted with compound of formula (XII-S) is NaOCH₃.

35. A process as in claim 30, wherein the source of (R)-epoxy-methylene is selected from the group consisting of (R)-glycidyl-m-nosylate and (R)-glycidyl tosylate.

36. A process as in claim 32, wherein the compound of formula (XIV-S) is de-protected by reacting the compound of formula (XIV) with hydrogen or a source of hydrogen.

37. A process as in claim 30, wherein the organic or inorganic base reacted with the compound of formula (V-S) is NaOCH₃.

38. A product prepared according to the process of claim 11.

39. A product prepared according to the process of claim 30.

40. A pharmaceutical composition comprising a pharmaceutically acceptable carrier and the product of claim 39.

41. A pharmaceutical composition made by mixing the product of claim 39 and a pharmaceutically acceptable carrier.

42. A process for making a pharmaceutical composition comprising mixing the product of claim 39 and a pharmaceutically acceptable carrier.

43. A method of treating epilepsy or a related disorder comprising administering to a subject in need thereof a therapeutically effective amount of the product of claim 39.

44. The method of claim 43, wherein the disorder is epilepsy.

45. A process for the preparation of a compound of formula (V) 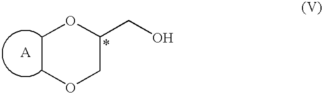
wherein 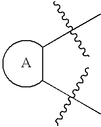
is selected from the group consisting of 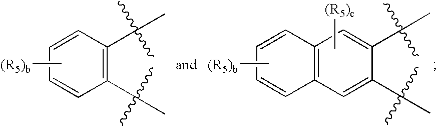
b is an integer from 0 to 4; and wherein c is an integer from 0 to 2;
each R⁵ is independently selected from the group consisting of halogen, lower alkyl and nitro;
comprising 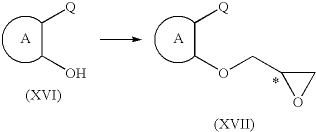
reacting a compound of formula (XVI) wherein Q is selected from the group consisting of —C(O)—(C₁₋₄alkyl); with a source of epoxy-methylene; in the presence of an inorganic base; at a temperature greater than about room temperature; in an organic solvent; to yield the corresponding compound of formula (XVII); 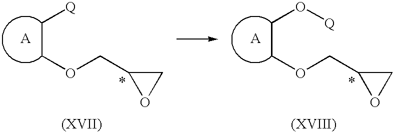
reacting the compound of formula (XVII) with an oxidizing agent; in an organic solvent; to yield the corresponding compound of formula (XVIII); 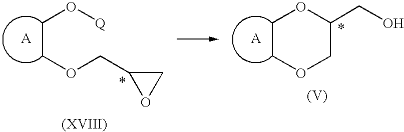
reacting the compound of formula (XVIII) with an organic or inorganic base; in a organic solvent; to yield the corresponding compound of formula (V).

46. A process as in claim 45, wherein Q is —C(O)—CH₃.

47. A process as in claim 45, wherein the source of epoxy-methylene is selected from the group consisting of glycidyl-m-nosylate and glycidyl tosylate.

48. A process as in claim 47, wherein the source of epoxy-methylene is selected from the group consisting of (R)-glycidyl-m-nosylate and (R)-glycidyl tosylate.

49. A process as in claim 45, wherein the oxidizing agent is m-CPBA.

50. A process as in claim 45, wherein the organic or inorganic acid reacted with the compound of formula (XVIII) is NaOCH₃.

51. A process as in claim 45, wherein

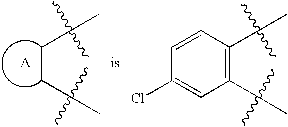

52. A process for the preparation of a compound of formula (I) 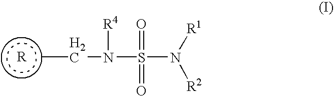
wherein 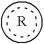
is selected from the group consisting of 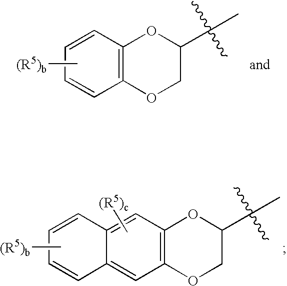
b is an integer from 0 to 4; and wherein c is an integer from 0 to 2;
each R⁵ is independently selected from the group consisting of halogen, lower alkyl and nitro;
R⁴ is selected from the group consisting of hydrogen and lower alkyl;
R¹ and R² are each independently selected from the group consisting of hydrogen and lower alkyl;
or pharmaceutically acceptable salts thereof; comprising 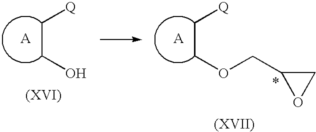
reacting a compound of formula (XVI) wherein 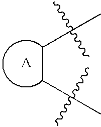
is selected from the group consisting of 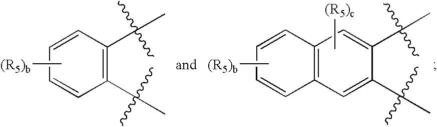
and wherein Q is selected from the group consisting of —C(O)—(C₁₋₄alkyl); with a source of epoxy-methylene; in the presence of an inorganic base; at a temperature greater than about room temperature; in an organic solvent; to yield the corresponding compound of formula (XVII); 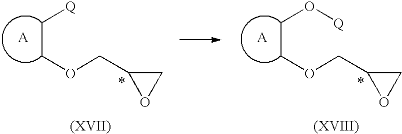
reacting the compound of formula (XVII) with an oxidizing agent; in an organic solvent; to yield the corresponding compound of formula (XVIII); 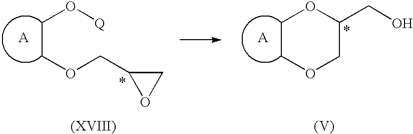
reacting the compound of formula (XVIII) with an organic or inorganic base; in a organic solvent; to yield the corresponding compound of formula (V); 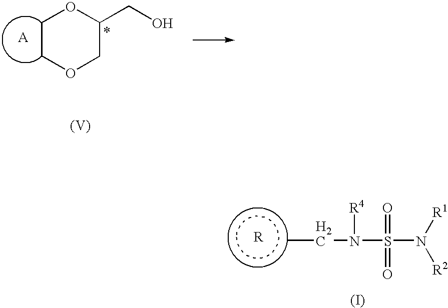
reacting the compound of formula (V); to yield the corresponding compound of formula (I).

53. A process as in claim 52, wherein Q is —C(O)—CH₃.

54. A process as in claim 52, wherein the source of epoxy-methylene is selected from the group consisting of glycidyl-m-nosylate and glycidyl tosylate.

55. A process as in claim 54, wherein the source of epoxy-methylene is selected from the group consisting of (R)-glycidyl-m-nosylate and (R)-glycidyl tosylate.

56. A process as in claim 52, wherein the oxidizing agent is m-CPBA.

57. A process as in claim 52, wherein the organic or inorganic acid reacted with the compound of formula (XVIII) is NaOCH₃.

58. A process as in claim 52, wherein 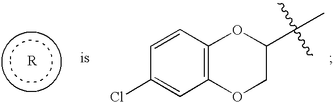
R¹ is hydrogen, R² is hydrogen and R⁴ is hydrogen.

59. A process for the preparation of the compound of formula (V-S) 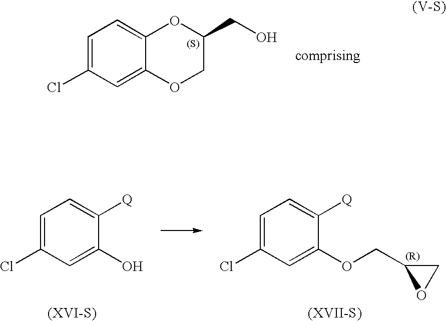
reacting a compound of formula (XVI-S) wherein Q is selected from the group consisting of —C(O)—(C₁₋₄alkyl); with a source of (R)-epoxy-methylene; in the presence of an inorganic base; at a temperature greater than about room temperature; in an organic solvent; to yield the corresponding compound of formula (XVII-S); 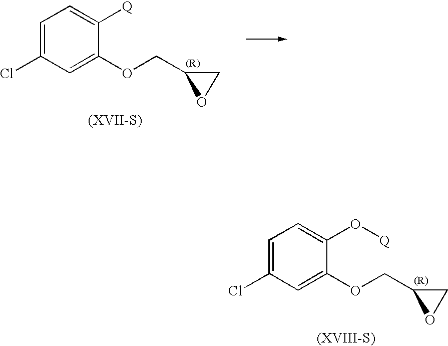
reacting the compound of formula (XVII-S) with an oxidizing agent; in an organic solvent; to yield the corresponding compound of formula (XVIII-S); 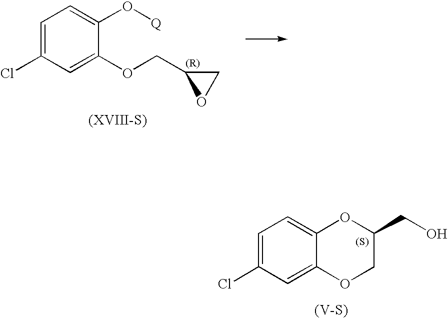
reacting the compound of formula (XVIII-S) with an organic or inorganic base; in a organic solvent; to yield the corresponding compound of formula (V-S).

60. A process as in claim 15, wherein Q is —C(O)—CH₃.

61. A process as in claim 15, wherein the source of (R)-epoxy-methylene is selected from the group consisting of (R)-glycidyl-m-nosylate and (R)-glycidyl tosylate.

62. A process as in claim 15, wherein the oxidizing agent is m-CPBA.

63. A process as in claim 15, wherein the organic or inorganic acid reacted with the compound of formula (XVIII-S) is NaOCH₃.

64. A process for the preparation of a compound of formula (I-S) 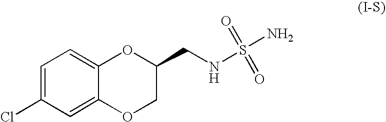
or a pharmaceutically acceptable salt thereof comprising 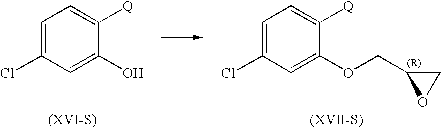
reacting a compound of formula (XVI-S) wherein Q is selected from the group consisting of —C(O)—(C₁₋₄alkyl); with a source of (R)-epoxy-methylene; in the presence of an inorganic base; at a temperature greater than about room temperature; in an organic solvent; to yield the corresponding compound of formula (XVII-S); 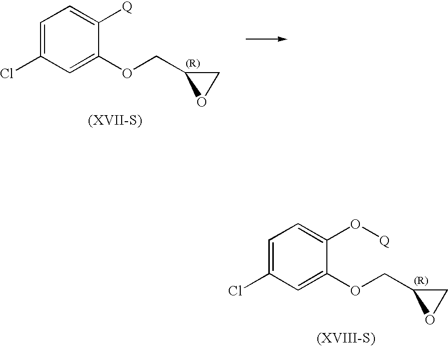
reacting the compound of formula (XVII-S) with an oxidizing agent; in an organic solvent; to yield the corresponding compound of formula (XVIII-S); 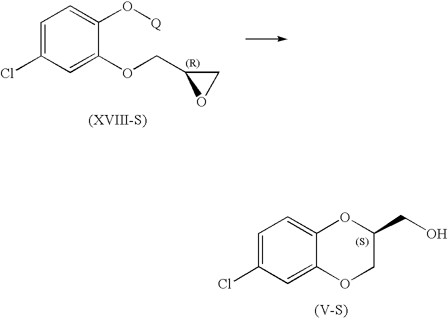
reacting the compound of formula (XVII I-S) with an organic or inorganic base; in a organic solvent; to yield the corresponding compound of formula (V-S); 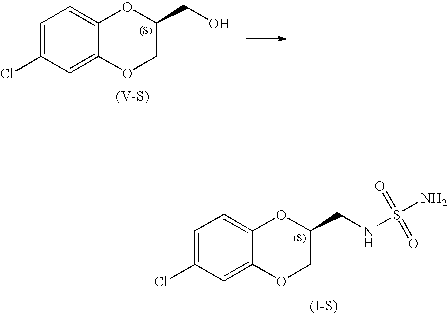
reacting the compound of formula (V-S); to yield the corresponding compound of formula (I-S).

65. A process as in claim 64, wherein Q is —C(O)—CH₃.

66. A process as in claim 64, wherein the source of (R)-epoxy-methylene is selected from the group consisting of (R)-glycidyl-m-nosylate and (R)-glycidyl tosylate.

67. A process as in claim 64, wherein the oxidizing agent is m-CPBA.

68. A process as in claim 64, wherein the organic or inorganic acid reacted with the compound of formula (XVIII-S) is NaOCH₃.

69. A product prepared according to the process of claim 52.

70. A product prepared according to the process of claim 64.

71. A pharmaceutical composition comprising a pharmaceutically acceptable carrier and the product of claim 70.

72. A pharmaceutical composition made by mixing the product of claim 72 and a pharmaceutically acceptable carrier.

73. A process for making a pharmaceutical composition comprising mixing the product of claim 70 and a pharmaceutically acceptable carrier.

74. A method of treating epilepsy or a related disorder comprising administering to a subject in need thereof a therapeutically effective amount of the product of claim 70.

75. The method of claim 76, wherein the disorder is epilepsy.

76. Crystalline form I-SA of the compound of formula (I-S)

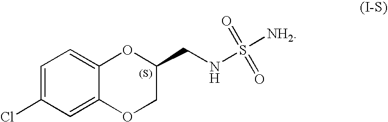

77. The crystalline form I-SA where in the crystalline form I-SA has the following powder X-ray diffraction peaks

| Position [°2θ] | d-spacing [Å] |
|---|---|
| 4.50 | 19.62 |
| 15.57 | 5.69 |
| 17.38 | 5.10 |
| 18.63 | 4.76 |
| 19.97 | 4.45 |
| 20.96 | 4.24 |
| 21.62 | 4.11 |
| 22.01 | 4.04 |
| 23.97 | 3.71 |
| 25.08 | 3.55 |
| 26.91 | 3.31 |
| 28.35 | 3.15 |
| 30.76 | 2.91 |

78. Crystalline form I-SB of the compound of formula (I-S)

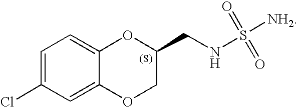

79. The crystalline form I-SB of claim 78 wherein the crystalline form I-SA has the following powder X-ray diffraction peaks:

| Position [° 2θ] | d-spacing [Å] |
|---|---|
| 4.48 | 19.74 |
| 8.91 | 9.92 |
| 13.36 | 6.62 |
| 17.84 | 4.97 |
| 18.61 | 4.77 |
| 22.33 | 3.98 |
| 26.86 | 3.32 |

80. A pharmaceutical composition comprising a pharmaceutically acceptable carrier and the product of claim 76.

81. A pharmaceutical composition made by mixing the product of claim 76 and a pharmaceutically acceptable carrier.

82. A process for making a pharmaceutical composition comprising mixing the product of claim 76 and a pharmaceutically acceptable carrier.

83. A method of treating epilepsy or a related disorder comprising administering to a subject in need thereof a therapeutically effective amount of the product of claim 76.

84. The method of claim 83, wherein the disorder is epilepsy.

85. A pharmaceutical composition comprising a pharmaceutically acceptable carrier and the product of claim 78.

86. A pharmaceutical composition made by mixing the product of claim 78 and a pharmaceutically acceptable carrier.

87. A process for making a pharmaceutical composition comprising mixing the product of claim 78 and a pharmaceutically acceptable carrier.

88. A method of treating epilepsy or a related disorder comprising administering to a subject in need thereof a therapeutically effective amount of the product of claim 78.

89. The method of claim 88, wherein the disorder is epilepsy.
